SEQUENCE LISTING (26 sequences)

SEQ ID NO: 1 - protein, 211 aa, Human
MSTAAVPELKQISRVEAMRLGPGWSHSCHAMLYAANPGQLFGRIPMRFSVLMQMRFDGLLGFPGGFVDRRFWSLEDGLNRVLGLGLGCLRLTEADYLSSHLTEGPHRVVAHLYARQLTLEQLHAVEISAVHSRDHGLEVLGLVRVPLYTQKDRVGGFPNFLSNAFVSTAKCQLLFALKVLNMMPEEKLVEALAAATEKQKKALEKLLPASS

SEQ ID NO: 2 - protein, 391 aa, Human
MESETEPEPVTLLVKSPNQRHRDLELSGDRGWSVGHLKAHLSRVYPERPRPEDQRLIYSGKLLLDHQCLRDLLPKQEKRHVLHLVCNVKSPSKMPEINAKVAESTEEPAGSNRGQYPEDSSSDGLRQREVLRNLSSPGWENISRPEAAQQAFQGLGPGFSGYTPYGWLQLSWFQQIYARQYYMQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 3 - protein, 389 aa, Human
MPSYTVTVATGSQWFAGTDDYIYLSLVGSAGCSEKHLLDKPFYNDFERGAVDSYDVTVDEELGEIQLVRIEKRKYWLNDDWYLKYITLKTPHGDYIEFPCYRWITGDVEVVLRDGRAKLARDDQIHILKQHRRKELETRQKQYRWMEWNPGFPLSIDAKCHKDLPRDIQFDSEKGVDFVLNYSKAMENLFINRFMHMFQSSWNDFADFEKIFVKISNTISERVMNHWQEDLMFGYQFLNGCNPVLIRRCTELPEKLPVTTEMVECSLERQLSLEQEVQQGNIFIVDFELLDGIDANKTDPCTLQFLAAPICLLYKNLANKIVPIAIQLNQIPGDENPIFLPSDAKYDWLLAKIWVRSSDFHVHQTITHLLRTHLVSEVFGIAMYRQLPA

SEQ ID NO: 4 - DNA, 633 bp, Human
atgtcgacggcggcggttccggagctgaagcagatcagccgggtggaggcgatgcgcctagggccgggctggagccactcgtgccacgccatgctgtacgccgccaaccctgggcagctcttcggccgcatccccatgcgcttctcggtgctgatgcagatgcgtttcgacgggctgctgggcttccccgggggcttcgtggaccggcgcttctggtcgctggaggacggcctgaaccgggtgctgggcctgggcctgggctgcctgcgcctcaccgaggccgactacctgagctcgcacctgaccgagggcccacaccgcgtcgtggcgcacctgtacgcgcggcagctgacgctggagcagctgcacgccgtggagatcagcgcggtgcactcgcgcgaccacggcctggaggtgctgggcctcgtgcgggtcccgctgtacacccagaaggaccgagtcggaggcttccccaacttcctgagcaacgccttcgtgagcacggctaagtgccagctcctctttgccctcaaggtgctcaacatgatgcccgaggagaagctggttgaggccctggctgcagccaccgagaagcagaagaaggccctggagaagttgctcccggcctcctct

SEQ ID NO: 5 - DNA, 1173 bp, Human
atggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaac

SEQ ID NO: 6 - DNA, 1167 bp, Human
atgccctcctacacggtcaccgtggccactggcagccagtggttcgccggcactgacgactacatctacctcagcctcgtgggctcggcgggctgcagcgagaagcacctgctggacaagcccttctacaacgacttcgagcgtggcgcggtggattcatacgacgtgactgtggacgaggaactgggcgagatccagctggtcagaatcgagaagcgcaagtactggctgaatgacgactggtacctgaagtacatcacgctgaagacgccccacggggactacatcgagttcccctgctaccgctggatcaccggcgatgtcgaggttgtcctgagggatggacgcgcaaagttggcccgagatgaccaaattcacattctcaagcaacaccgacgtaaagaactggaaacacggcaaaaacaatatcgatggatggagtggaaccctggcttccccttgagcatcgatgccaaatgccacaaggatttaccccgtgatatccagtttgatagtgaaaaaggagtggactttgttctgaattactccaaagcgatggagaacctgttcatcaaccgcttcatgcacatgttccagtcttcttggaatgacttcgccgactttgagaaaatctttgtcaagatcagcaacactatttctgagcgggtcatgaatcactggcaggaagacctgatgtttggctaccagttcctgaatggctgcaaccctgtgttgatccggcgctgcacagagctgcccgagaagctcccggtgaccacggagatggtagagtgcagcctggagcggcagctcagcttggagcaggaggtccagcaagggaacattttcatcgtggactttgagctgctggatggcatcgatgccaacaaaacagacccctgcacactccagttcctggccgctcccatctgcttgctgtataagaacctggccaacaagattgtccccattgccatccagctcaaccaaatcccgggagatgagaaccctattttcctcccttcggatgcaaaatacgactggcttttggccaaaatctgggtgcgttccagtgacttccacgtccaccagaccatcacccaccttctgcgaacacatctggtgtctgaggtttttggcattgcaatgtaccgccagctgcctgct

SEQ ID NO: 7 - protein, 53 aa, Human
MDAEFRHDSGYEVHHQKLVFFAEDVGSNKGAIIGLMVGGVVIATVIVITLVML

SEQ ID NO: 8 - DNA, 39 bp, Artificial Sequence
  Primer
atctggtaccccaccatggatgcagaattccgacatgac

SEQ ID NO: 9 - DNA, 33 bp, Artificial Sequence
  Primer
gcttctagacagcatcaccaaggtgatgacgat

SEQ ID NO: 10 - protein, 786 aa, Human
LSRKRRRQHGQLWFPEGFKVSEASKKKRREPLGEDSVGLKPLKNASDGALMDDNQNEWGDEDLETKKFRFEEPVVLPDLSDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDRRKRLEASADANIQDNMGRTPLHAAVSADAQGVFQILLRNRATDLDARMHDGTTPLILAARLAVEGMLEDLINSHADVNAVDDLGKSALHWAAAVNNVDAAVVLLKNGANKDIENNKEETSLFLSIRRESYETAKVLLDHFANRDITDHMDRLPRDIAQERMHHDIVRLLDEYNLVRSPQLHGTALGGTPTLSPTLCSPNGYPGNLKSATQGKKARKPSTKGLACGSKEAKDLKARRKSSQDGKGWLLDSSSSMLSPVDSLESPHGYLSDVASHPLLPSPFQQSPSMPLSHLPGMPDTHLGISHLNVAAKPEMAALAGGSRLAFEHPPPRLSHLPVASSACTVLSTNGTGAMNFTVGAPASLNGQCEWLPRLQNGMVPSQYNPLRPGVTPGTLSTQAAGLQHSMMGPLHSSLSTNTLSPIIYQGLPNTRLATQPHLVQTQQVQPQNLPLQPQNLQPPSQPHLSVSSAANGHLGRSFLSGEPSQADVQPLGPSSLPVHTILPQESQALPTSLPSSMVPPMTTTQFLTPPSQHSYSSSPVDNTPSHQLQVPEPTFLTPSPESPDQWSSSSPHSNISDWSEGISSPPTTMPSQITHIPEAFK

SEQ ID NO: 11 - DNA, 33 bp, Artificial Sequence
  Primer
ctgtctagaaagcgccggcgccagcatggccag

SEQ ID NO: 12 - DNA, 24 bp, Artificial Sequence
  Primer
attgttcaccgcggccgcccaatg

SEQ ID NO: 13 - DNA, 33 bp, Artificial Sequence
  DNA oligomer
aattcggtacccccggggcggccgcctcgagga

SEQ ID NO: 14 - DNA, 33 bp, Artificial Sequence
  DNA oligomer
gccatgggggccccgccggcggagctcctttaa

SEQ ID NO: 15 - DNA, 354 bp, Human
ctcaggcgcgcgccattggccgccagaccttgtgcctagcggccaatgggggggcgcagtccacgagcggtgccgcgtgtctcttcctcccattggctgaaagttactgtgggaaagaaagtttgggaagtttcacacgagccgttcgcgtgcagtcccagatatatatagaggccgccagggcctgcggatcacacaggatctggagctggtgctgataacagcggaatcccctgtctacctctctccttggtcctggaatagtgctaccgatcactaagtagccctaagacataataaaccttcaactgctcagtagtttttcttatgaaagtcaagtaaaaggacgtaagc

SEQ ID NO: 16 - DNA, 21 bp, Artificial Sequence
  Primer
ggtggaccatcctctagactg

SEQ ID NO: 17 - DNA, 20 bp, Artificial Sequence
  Primer
gttacttaagctagcttgcc

SEQ ID NO: 18 - protein, 40 aa, Human
DAEFRHDSGYEVHHQKLVFFAEDVGSNKGAIIGLMVGGVV

SEQ ID NO: 19 - protein, 42 aa, Human [sequence omitted]

SEQ ID NO: 20 - protein, 52 aa, Human
DAEFRHDSGYEVHHQKLVFFAEDVGSNKGAIIGLMVGGVVIATVIVITLVML

SEQ ID NO: 21 - DNA, 2076 bp, Human
cgcgccatgccctcctacacggtcaccgtggccactggcagccagtggttcgccggcactgacgactacatctacctcagcctcgtgggctcggcgggctgcagcgagaagcacctgctggacaagcccttctacaacgacttcgagcgtggcgcggtggattcatacgacgtgactgtggacgaggaactgggcgagatccagctggtcagaatcgagaagcgcaagtactggctgaatgacgactggtacctgaagtacatcacgctgaagacgccccacggggactacatcgagttcccctgctaccgctggatcaccggcgatgtcgaggttgtcctgagggatggacgcgcaaagttggcccgagatgaccaaattcacattctcaagcaacaccgacgtaaagaactggaaacacggcaaaaacaatatcgatggatggagtggaaccctggcttccccttgagcatcgatgccaaatgccacaaggatttaccccgtgatatccagtttgatagtgaaaaaggagtggactttgttctgaattactccaaagcgatggagaacctgttcatcaaccgcttcatgcacatgttccagtcttcttggaatgacttcgccgactttgagaaaatctttgtcaagatcagcaacactatttctgagcgggtcatgaatcactggcaggaagacctgatgtttggctaccagttcctgaatggctgcaaccctgtgttgatccggcgctgcacagagctgcccgagaagctcccggtgaccacggagatggtagagtgcagcctggagcggcagctcagcttggagcaggaggtccagcaagggaacattttcatcgtggactttgagctgctggatggcatcgatgccaacaaaacagacccctgcacactccagttcctggccgctcccatctgcttgctgtataagaacctggccaacaagattgtccccattgccatccagctcaaccaaatcccgggagatgagaaccctattttcctcccttcggatgcaaaatacgactggcttttggccaaaatctgggtgcgttccagtgacttccacgtccaccagaccatcacccaccttctgcgaacacatctggtgtctgaggtttttggcattgcaatgtaccgccagctgcctgctgtgcaccccattttcaagctgctggtggcacacgtgagattcaccattgcaatcaacaccaaggcccgtgagcagctcatctgcgagtgtggcctctttgacaaggccaacgccacagggggcggtgggcacgtgcagatggtgcagagggccatgaaggacctgacctatgcctccctgtgctttcccgaggccatcaaggcccggggcatggagagcaaagaagacatcccctactacttctaccgggacgacgggctcctggtgtgggaagccatcaggacgttcacggccgaggtggtagacatctactacgagggcgaccaggtggtggaggaggacccggagctgcaggacttcgtgaacgatgtctacgtgtacggcatgcggggccgcaagtcctcaggcttccccaagtcggtcaagagccgggagcagctgtcggagtacctgaccgtggtgatcttcaccgcctccgcccagcacgccgcggtcaacttcggccagtacgactggtgctcctggatccccaatgcgcccccaaccatgcgagccccgccaccgactgccaagggcgtggtgaccattgagcagatcgtggacacgctgcccgaccgcggccgctcctgctggcatctgggtgcagtgtgggcgctgagccagttccaggaaaacgagctgttcctgggcatgtacccagaagagcattttatcgagaagcctgtgaaggaagccatggcccgattccgcaagaacctcgaggccattgtcagcgtgattgctgagcgcaacaagaagaagcagctgccatattactacttgtccccagaccggattccgaacagtgtggccatctgagcacactgccagtctcactgtgggaaggccagctgccccagccag

SEQ ID NO: 22 - protein, 674 aa, Human
MPSYTVTVATGSQWFAGTDDYIYLSLVGSAGCSEKHLLDKPFYNDFERGAVDSYDVTVDEELGEIQLVRIEKRKYWLNDDWYLKYITLKTPHGDYIEFPCYRWITGDVEVVLRDGRAKLARDDQIHILKQHRRKELETRQKQYRWMEWNPGFPLSIDAKCHKDLPRDIQFDSEKGVDFVLNYSKAMENLFINRFMHMFQSSWNDFADFEKIFVKISNTISERVMNHWQEDLMFGYQFLNGCNPVLIRRCTELPEKLPVTTEMVECSLERQLSLEQEVQQGNIFIVDFELLDGIDANKTDPCTLQFLAAPICLLYKNLANKIVPIAIQLNQIPGDENPIFLPSDAKYDWLLAKIWVRSSDFHVHQTITHLLRTHLVSEVFGIAMYRQLPAVHPIFKLLVAHVRFTIAINTKAREQLICECGLFDKANATGGGGHVQMVQRAMKDLTYASLCFPEAIKARGMESKEDIPYYFYRDDGLLVWEAIRTFTAEVVDIYYEGDQVVEEDPELQDFVNDVYVYGMRGRKSSGFPKSVKSREQLSEYLTVVIFTASAQHAAVNFGQYDWCSWIPNAPPTMRAPPPTAKGVVTIEQIVDTLPDRGRSCWHLGAVWALSQFQENELFLGMYPEEHFIEKPVKEAMARFRKNLEAIVSVIAERNKKKQLPYYYLSPDRIPNSVAI

SEQ ID NO: 23 - DNA, 29 bp, Artificial Sequence
  primer
cggaattccgcgccatgccctcctacacg

SEQ ID NO: 24 - DNA, 24 bp, Artificial Sequence
  primer
ccccgcatgccgtacacgtagaca

SEQ ID NO: 25 - DNA, 24 bp, Artificial Sequence
  primer
tgtctacgtgtacggcatgcgggg

SEQ ID NO: 26 - DNA, 32 bp, Artificial Sequence
  primer
gcgtcgacctggctggggcagctggccttccc

(19) United States
(12) Patent Application Publication
(10) Pub. No.: US 2004/0234990 A1
(43) Pub. Date: Nov. 25, 2004

(54) Method of screening alzheimer's disease-associated gene

(76) Inventor: Hiroto Komano, Aichi (JP)

Correspondence Address: EDWARDS & ANGELL, LLP, P.O. BOX 55874, BOSTON, MA, 02205, US

(21) Appl. No.: 10/485,310

(22) PCT Filed: Feb. 1, 2002
(86) PCT No.: PCT/JP02/00836

(30) Foreign Application Priority Data
Jul. 31, 2001 (JP) 2001-266510

(51) Int. Cl. C12Q 1/68; C07H 21/04

(52) U.S. Cl. 435/6; 536/24.3

(57) ABSTRACT

The present invention aims at providing a method of screening a gene that controls the production of amyloid β protein. More specifically, the present invention provides a gene that controls the production of amyloid β protein, a protein encoded by this gene or its salt, a screening method by using the gene, an antibody to the above-described protein, diagnostic agent comprising the antibody, etc.

### TECHNICAL FIELD

The present invention relates to a method for investigations of a gene that controls the production of amyloid β protein (Aβ), which is deeply associated with the onset/progression of Alzheimer's disease. That is, the present invention relates to a cell line designed to increase the production of Aβ from Aβ precursor protein (βAPP) thereby to activate the expression of a drug-resistant gene, and a recombinant DNA construct necessary for producing the cell line, as well as a method of screening a gene that controls the production of Aβ by identifying cDNA of the cell, which comprises transfecting a human cDNA library to the cell line and identifying cDNA of the cell which acquires the drug-resistant ability, thus increasing the production of Aβ. Furthermore, the present invention relates to a gene that controls the production of Aβ found by this screening method, a recombinant vector bearing the gene, a transformant using the recombinant vector, a process of manufacturing a recombinant protein using the transformant, a recombinant protein manufactured by the process, an antibody to the recombinant protein, a method of screening an Aβ production inhibitor by using the transformant or recombinant protein, and an Aβ production inhibitor obtained by the screening method. The present invention further relates to these genes, nucleotides specifically hybridizable to the genes, recombinant proteins derived from these genes, pharmaceutical compositions comprising the antibody or Aβ production inhibitor, and a method for diagnosis, treatment and prevention of diseases.

### BACKGROUND ART

Pathological features in the brain from patients with Alzheimer's disease are known to be senile plaques and accumulation of neurofibrillary tangles, in addition to neuronal loss. Among them, the pathological hallmark at the initial stage is the formation of senile plaques, and a major component in the senile plaques is Aβ.

Thus, it is considered that abnormalities in the production or degradation of Aβ would be closely associated with the onset/progression of Alzheimer's disease. Aβ is cleaved from the Aβ precursor protein (βAPP) by β-secretase and γ-secretase and produced. With regard to the β-secretase, it was already identified to be a novel aspartic protease (Neuron, 27, 419-422, 2000). Turning to the γ-secretase, it has been revealed that a familial Alzheimer's disease (FAD)-pathogenic gene, presenilin or a complex containing presenilin takes part in exhibiting its activity (Neuron, 27, 419-422, 2000). And recently, Nicastrin was reported to be a new transmembrane glycoprotein, which is engaged with processing of βAPP (Nature, 407, 48-54, 2000).

However, any substantial detail of the γ-secretase is unclear (Neuron, 27, 419-422, 2000). The γ-secretase activity is recovered in a macromolecular fraction by anti-presenilin antibodies, and it is perceived that many unidentified factors including stabilizing factors for the complex of presenilin N-terminal and C-terminal fragments, etc. could participate in controlling the activity. Furthermore, the processing of βAPP generates in the rough-surfaced endoplasmic reticulum, on the secretory pathway trafficking from the Golgi body, etc. to the cell membrane, on the cell membrane and further in the endosomes after βAPP is reinternalized into the cells (Trends in Cell Biology, 8, 447-453, 1998). It is thus considered that various factors associated with the intracellular trafficking system of βAPP could affect the production of Aβ as well. Thus, it is considered extremely important to identify these factors as genes, because such will lead to application to new pharmaceuticals including the development of drugs to prevent the production of Aβ, diagnostics for Alzheimer's disease, etc.

### DISCLOSURE OF THE INVENTION

In order to investigate a gene that controls the production of Aβ, the present inventor first contrived a cell line designed to increase the production of Aβ from the βAPP fragment thereby to activate the expression of a drug-resistant gene. More specifically, the inventor first prepared the βAPP fragment containing the γ-secretase cleavage site of βAPP and a fusion gene encoding a fusion protein (CAPP-NICD) to the Notch C-terminal transcription factor domain; second, prepared a selection marker gene bound to puromycin-N-acetyltransferase gene downstream of HES-1 promoter so as to initiate transcription of a puromycin-resistant gene by the transcription activity of Notch; and then prepared a cell line wherein the first and second recombinant genes were co-expressed. That is, this cell line is so designed that CAPP-NICD undergoes intracellular cleavage by γ-secretase to release the Notch transcription factor from the cell membrane, whereby the cells are rendered resistant to puromycin. In fact, the inventor verified that in the cells Aβ is produced from CAPP-NICD by endogenous γ-secretase and depending thereon, a puromycin-resistant gene is expressed. Accordingly, identification of cDNA for the regulatory factor to promote γ-secretase or γ-secretase activity using the cells can be made by the following 4 steps. First, human cDNA library is transfected to the cell line, and cells bearing DNAs having increased puromycin resistance are selected. Next, the Aβ produced by these cells are measured, and cDNAs that increase the production of Aβ are selected. The cDNAs are further identified by PCR. Last, the cDNAs obtained are transfected to other cells wherein APP is expressed to examine if the resulting cDNAs increase the production of Aβ. By these means, the inventor has so far identified at least 3 genes as the regulatory factor to activate γ-secretase or γ-secretase activity. Based on these findings, the present inventors have made extensive investigations and come to accomplish the present invention.

Thus, the present invention provides the following features:

(1) A method of screening a DNA that controls the production of amyloid β protein (Aβ), which comprises transfecting a DNA library to the cell line designed to activate the expression of a selection marker gene by the increased production of Aβ from the Aβ precursor protein (βAPP) fragment, and identifying a DNA clone transfected with the cell in which the production of Aβ is increased;

(2) The screening method according to (1), wherein the DNA that controls the production of Aβ is human cDNA;

(3) The screening method according to (1), wherein the DNA that controls the production of Aβ is human chromosomal DNA;

(4) The screening method according to (1), wherein the cell line is a transformant transformed by (1) a vector bearing a DNA encoding a fusion protein of the βAPP fragment containing the γ-secretase cleavage site of βAPP and a transcription-promoting factor, and (2) a vector bearing a DNA ligated to the selection marker gene downstream of the promoter to induce transcription of the selection marker gene by the transcription activity of transcription-promoting factor,

(5) The screening method according to (1), wherein the cell line is a transformant transformed by (1) a vector bearing a DNA encoding a fusion protein (CAPP-NICD) of the βAPP fragment containing the γ-secretase cleavage site of βAPP and the C-terminal transcription factor domain of Notch, and (2) a vector bearing a DNA ligated to a drug-resistant gene downstream of HES-1 promoter to induce transcription of drug-resistant gene by the transcription activity of Notch;

(6) The screening method according to (4) or (5), wherein the cell line co-expresses the fusion protein and the selection marker,

(7) A DNA containing a DNA hybridizable to the DNA that controls the production of Aβ under high stringent conditions which is obtainable by the screening method according to (1);

(8) The DNA according to (7), wherein the DNA that controls the production of Aβ is human cDNA;

(9) The DNA according to (7), wherein the DNA that controls the production of Aβ is human chromosomal DNA;

(10) The DNA according to (7), which is associated with Alzheimer's disease;

(11) The DNA according to (7), which is a DNA that increases the production of Aβ;

(12) The DNA according to (11), wherein the DNA that increases the production of Aβ is a DNA encoding human cDNA (Genbank accession No. AAH06223) containing the base sequence represented by SEQ ID NO: 4;

(13) The DNA according to (11), wherein the DNA that increases the production of Aβ is a DNA encoding human Herp (Genbank accession No. AB034989) containing the base sequence represented by SEQ ID NO: 5;

(14) The DNA according to (11), wherein the DNA that increases the production of Aβ is a DNA encoding human 5-lipoxygenase (Genbank accession no. XM 005818) containing the base sequence represented by SEQ ID NO: 6;

(15) The DNA according to (11), wherein the DNA that increases the production of Aβ is a DNA encoding the full-length sequence of human 5-lipoxygenase containing the base sequence represented by SEQ ID NO: 21;

(16) A recombinant vector comprising the DNA according to (7);

(17) A transformant transformed by the recombinant vector according to (16);

(18) A process of manufacturing a peptide or protein encoded by the DNA according to (7), or a salt thereof, which comprises culturing the transformant according to (17) and producing the peptide or protein encoded by the DNA according to (7);

(19) A diagnostic product comprising the DNA according to (7) or a part thereof;

(20) The diagnostic product according to (19), which is a diagnostic product for Alzheimer's disease;

(21) A method for diagnosis of Alzheimer's disease, which comprises using the DNA according to (7);

(22) A method for detection of single nucleotide polymorphisms (SNPs), which comprises using the DNA according to (7);

(23) The method for detection according to (22), which comprises decoding the base sequence of a DNA corresponding to the DNA according to (7) of a patient with Alzheimer's disease and comparing the decoded base sequence with that of the DNA according to (7);

(24) Single nucleotide polymorphisms (SNPs) of the DNA according to (7);

(25) A diagnostic product comprising the single nucleotide polymorphisms (SNPs) according to (24);

(26) The diagnostic product according to (25), which further contains the DNA according to (7) or a part thereof;

(27) The diagnostic product according to (25) or (26), which is a diagnostic product for Alzheimer's disease;

(28) A method for diagnosis of Alzheimer's disease, which comprises using single nucleotide polymorphisms (SNPs) according to (24);

(29) The method for diagnosis according to (28), wherein the DNA according to (7) or a part thereof is used;

(30) A peptide or protein encoded by the DNA according to (7), or a salt thereof;

(31) The peptide, protein or its salt according to (30), which is associated with Alzheimer's disease;

(32) The peptide, protein or its salt according to (30), which increases the production of Aβ;

(33) A protein that increases the production of Aβ, or a salt thereof, containing the same or substantially the same amino acid sequence as the amino acid sequence represented by SEQ ID NO: 1;

(34) A protein that increases the production of Aβ, or a salt thereof, containing the same or substantially the same amino acid sequence as the amino acid sequence represented by SEQ ID NO: 2;

(35) A protein that increases the production of Aβ, or a salt thereof, containing the same or substantially the same amino acid sequence as the amino acid sequence represented by SEQ ID NO: 3;

(36) A protein that increases the production of Aβ, or a salt thereof, containing the same or substantially the same amino acid sequence as the amino acid sequence represented by SEQ ID NO: 22;

(37) An antibody to the peptide, protein or its salt according to any one of (29) through (36);

(38) The antibody according to (37), which is a neutralizing antibody for inactivating the activity of the peptide, protein or its salt according to any one of (29) through (36);

(39) A diagnostic product comprising the antibody according to (37);

(40) The diagnostic product according to (39), which is a diagnostic product for Alzheimer's disease;

(41) A method for quantification of the peptide, protein or its salt according to any one of (29) through (36), which comprises using the antibody according to (37);

(42) A method for diagnosis of Alzheimer's disease, using the method for quantification according to (41);

(43) A pharmaceutical comprising an antibody to the peptide, protein or its salt according to (32), which increases the production of Aβ;

(44) The pharmaceutical according to (43), which is an agent for the prevention/treatment of Alzheimer's disease;

(45) A method of preventing/treating Alzheimer's disease, which comprises administering to a mammal an effective dose of the antibody to the peptide, protein or its salt according to (32) that increases the production of Aβ;

(46) An antisense DNA comprising a complementary base sequence to the DNA according to (7), or a part thereof;

(47) A diagnostic product comprising the antisense DNA according to (46);

(48) The diagnostic product according to (47), which is a diagnostic product for Alzheimer's disease;

(49) A pharmaceutical comprising an antisense DNA comprising a complementary base sequence to the DNA according to (11) that increases the production of Aβ, or a part thereof;

(50) The pharmaceutical according to (49), which is an agent for the prevention/treatment of Alzheimer's disease;

(51) A method of preventing/treating Alzheimer's disease, which comprises administering to a mammal an effective dose of the antisense DNA according to (49);

(52) A method of screening an Aβ production inhibitor, which comprises using the DNA according to (7);

(53) A method of screening an Aβ production inhibitor, which comprises using the peptide, protein or its salt according to (30);

(54) A method of screening an Aβ production inhibitor, which comprises using the antibody according to (37);

(55) A method of screening an Aβ production inhibitor, which comprises using the antisense DNA according to (46);

(56) A method of screening an Aβ production inhibitor, which comprises using the transformant according to (17);

(57) An Aβ production inhibitor, which is obtainable by the screening method according to any one of (52) through (56);

(58) A pharmaceutical comprising the Aβ production inhibitor according to (57);

(59) The pharmaceutical according to (58), which is an agent for the prevention/treatment of Alzheimer's disease;

(60) A method of preventing/treating Alzheimer's disease, which comprises administering to a mammal an effective dose of the Aβ production inhibitor according to (57),

(61) A method of screening a substance that controls the production of Aβ, which comprises measuring a difference between respective levels of Aβ produced, when a test compound is added to (i) the cell line designed to activate the expression of a selection marker gene by the increased production of Aβ from the Aβ precursor protein (βAPP) fragment, and to (ii) the cell line transfected with the DNA that increases the production of Aβ according to (11);

(62) A method of screening a substance that controls the production of Aβ, which comprises measuring a difference between respective biological activities of the selection marker, when a test compound is added to (i) the cell line designed to activate the expression of a selection marker gene by the increased Aβ production from the Aβ precursor protein (βAPP) fragment, and to (ii) the cell line transfected with the DNA that increases the production of Aβ according to (12);

(63) The screening method according to (62), wherein the selection marker gene is a drug-resistant gene and the biological activity of the selection marker is drug resistance;

(64) A method of screening an expression inhibitor of the DNA, which comprises using the DNA according to (7);

(65) A method of screening a compound that suppresses or promotes the promoter activity of a gene encoding the peptide or protein, which comprises assaying the respective reporter activities in combination of the promoter region in the human chromosomal DNA according to (9) with a reporter gene, where a test compound is added and where no test compound is added; and,

(66) A method of screening a compound that suppresses the respective expression levels of the peptide, protein or its salt according to (30), or DNAs thereof, when a test compound is added to a cell capable of expressing the said peptide, protein or its salt, and when no test compound is added thereto.

The present invention further provides the following features:

(67) A dominant negative peptide or protein for the peptide or protein to increase the production of Aβ according to (32), or a salt thereof;

(68) The dominant negative peptide, protein or its salt according to (67), wherein the activity of increasing the production of Aβ is lost or attenuated by the substitution, deletion or/and addition of amino acid sequence;

(69) A pharmaceutical comprising the dominant negative peptide, protein or its salt according to (67);

(70) The pharmaceutical according to (69), which is an agent for the prevention/treatment of Alzheimer's disease;

(71) A method of preventing/treating Alzheimer's disease, which comprises administering to a mammal an effective dose of the dominant negative peptide, protein or its salt according to (67);

(72) A DNA encoding the dominant negative peptide, protein or its salt according to (67);

(73) A pharmaceutical comprising the DNA according to (72);

(74) The pharmaceutical according to (73), which is an agent for the prevention/treatment of Alzheimer's disease; and,

(75) A method of preventing/treating Alzheimer's disease, which comprises administering to a mammal an effective dose of the DNA according to (72).

### BRIEF DESCRIPTION OF THE DRAWING

FIG. 1 shows a schematic view to examine the expression of puromycin-resistant gene in the Notch transcription factor domain liberated from C53NICD chimeric protein by γ-secretase.

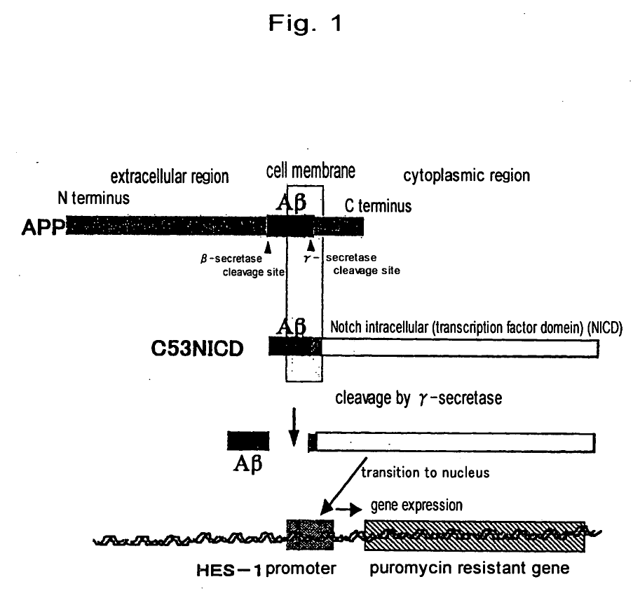

### BEST MODE FOR CARRYING OUT THE INVENTION

[DNA constructs]

In the method of investigating the DNA that controls the production of Aβ, there is used the cell line designed to increase the production of Aβ from the βAPP fragment thereby to activate the expression of a selection marker gene.

Two kinds of recombinant gene constructs are transfected to this cell line.

The first recombinant gene construct is a construct (vector) containing a fusion gene encoding the fusion protein of the βAPP fragment containing the γ-secretase cleavage site of βAPP having the amino acid sequence represented by SEQ ID NO: 7 and a protein that promotes a certain transcription factor activity (transcription-promoting factor).

The βAPP fragment may be of any fragment of any length, so long as it is a fragment that can anchor the fusion protein to the cell membrane, has a length sufficient to include Aβ40 (SEQ ID NO: 18) or/and Aβ42 (SEQ ID NO: 19) as the γ-secretase cleavages sites of βAPP and is cleaved by γ-secretase; Aβ (1-52) having the amino acid sequence represented by SEQ ID NO: 20 is preferably used. The βAPP fragment may also be deleted of, added with or substituted with 1 or more amino acids, so long as it is cleaved by γ-secretase. These substitutions may contain amino acid mutations found in familial Alzheimer's disease (FAD).

For the protein used in the fusion protein that promotes the transcription factor activity (transcription-promoting factor), it is required that the protein should be distinguishable from the endogenous transcription factor system either in quality or in quantity, the fusion protein to the βAPP fragment described above should be anchored to the cell membrane, and when the fusion protein is cleaved by γ-secretase, it should undergo nuclear translocation to promote transcription, etc. As long as these conditions are met, any transcription factor system is usable and, preferably the transcription factor domain or transcription promotion active domain of SREBP, Notch, Ire1 or ATF6, which is the transcription factor system receiving the regulated intramembrane proteolysis (Cell, 100, 391-396, 2000) is used, more preferably the intracellular domain of Notch, namely, the C-terminal transcription factor domain (NICD) having the amino acid sequence represented by SEQ ID NO: 10.

The second recombinant gene construct is a construct (vector) containing a promoter sequence where the transcription factor used for the fusion protein described above can act and a selection marker gene downstream.

The promoter sequence may take any sequence so long as its promoter activity is exhibited selectively to the target transcription-promoting factor. For example, when NICD is used as the transcription-promoting factor, HES-1 or its analogous sequence is used as the promoter sequence; when the RNase L domain of Ire1 is used as the transcription-promoting factor, unfolded protein response element (UPRE) is used as the promoter sequence; and, when p50ATF6 is used as the transcription-promoting factor, ER stress response element (ERSE) or its analogous sequence is used as the promoter sequence. The analogous sequences described above may contain one or more insertions, substitutions or deletions.

As the selection marker gene, any gene is usable as far as it is expressed under control of the promoter sequence and the expression is easily detectable, and a drug-resistant gene is preferably used (Shin Seikagaku Jikken Koza 2, Nucleic Acid III, 3.6 Animal Cell Expression Vector, pages 84-103).

For the combination of drug-resistant genes and drugs, the following examples can be used:

(1) combination of puromycin-N-acetyltransferase gene and puromycin;

(2) combination of aminoglycoside phosphotransferase gene (APH) and G418;

(3) combination of hygromycin B phosphotransferase gene (HPH) and hygromycin B;

(4) combination of xanthine-guanine phosphoribosyltransferase (XGPRT) and mycophenolic acid; etc.

Also, where the parent cell line is a hypoxanthine-guanine phosphoribosyltransferase (HGPRT) or thymidine kinase (TK)-deficient strain, these genes are used in combination with HAT (hypoxanthine, aminopterin, thymidine).

Furthermore, dihydrofolate reductase, ampicillin-resistant gene, etc. may be used as the selection marker gene. Various reporter genes may also be used in place of the selection marker gene described above. As such reporter genes, there may be preferably used chloramphenicol acetyltransferase (CAT), β-galactosidase, luciferase, growth factor, β-glcuronidase, alkaline phosphatase, Green fluorescent protein (GFP), β-lactamase, etc. Vector construction of these reporter genes and their assay methods may be carried out according to publicly known techniques (e.g., Molecular Biotechnology, 13, 29-43, 1999).

In addition to the foregoing, an enhancer, a splicing signal, a polyA-added signal, other selection markers, SV40 replication origin (hereinafter sometimes abbreviated as SV40ori), etc. may also be added to the vectors containing these first and second recombinant gene constructs.

[Cell Line]

The cell line used for the screening method of the present invention is not particularly limited, as far as it has the function to increase the production of Aβ from the βAPP fragment thereby to activate the expression of the selection marker gene. Specifically, the cell line transfected with the two recombinant gene constructs described above is used.

Any cell line, to which the said two recombinant gene constructs are transfected, may be used, so long as the cell line can efficiently transfect the DNA library. Desirably, the cell line is the one that βAPP is not expressed endogenously, but even if βAPP is expressed, such a cell line may also be used if the transfected βAPP fragment is efficiently cleaved. Moreover, even the cell line having the γ-secretase activity endogenously or the cell line transfected with presenilin, etc. thereby to acquire the γ-secretase activity may be used, so long as it has the γ-secretase activity sufficient to detect. Specific examples of the cell line include animal cell lines including monkey cell COS-7, Vero, Chinese hamster cell CHO, dhfr gene-deficient CHO, mouse L cells, mouse AtT-20, mouse myeloma cells, rat GH3, human FL cells, HEK-293 cells etc., and mouse pro-B cell-derived cell line, BaF/3, is preferably used.

The DNA in the DNA library may be any of cDNA, chromosomal DNA and synthetic DNA. The chromosomal DNA may contain a promoter region, an enhancer region, etc.

As the DNA library, those derived from human and other warm-blooded animals (e.g., guinea pig, rat, mouse, fowl, rabbit, swine, sheep, bovine, monkey, etc.) are employed; especially human-derived library is preferably employed.

The DNA library may be derived from any cells of human and other warm-blooded animals (e.g., retina cell, hepatocyte, splenocyte, nerve cell, glial cell, β cell of pancreas, bone marrow cell, mesangial cell, Langerhans' cell, epidermic cell, epithelial cell, endothelial cell, fibroblast, fibrocyte, myocyte, fat cell, immune cell (e.g., macrophage, T cell, B cell, natural killer cell, mast cell, neutrophil, basophil, eosinophil, monocyte), megakaryocyte, synovial cell, chondrocyte, bone cell, osteoblast, osteoclast, mammary gland cell, hepatocyte or interstitial cell, or the corresponding precursor cells, stem cells, cancer cells, etc.), or any tissues where such cells are present, such as brain or any of brain regions (e.g., retina, olfactory bulb, amygdaloid nucleus, basal ganglia, hippocampus, thalamus, hypothalamus, cerebral cortex, medulla oblongata, cerebellum), spinal cord, pituitary, stomach, pancreas, kidney, liver, gonad, thyroid, gall-bladder, bone marrow, adrenal gland, skin, muscle, lung, gastrointestinal tract (e.g., large intestine and small intestine), blood vessel, heart, thymus, spleen, submandibular gland, peripheral blood, prostate, testis, ovary, placenta, uterus, bone, joint, skeletal muscle, etc.; or those derived from hemocyte type cells or their cultured cells (e.g., MEL, M1, CTLL-2, HT-2, WEHI-3, HL-60, JOSK-1, K562, ML-1, MOLT-3, MOLT-4, MOLT-10, CCRF-CEM, TALL-1, Jurkat, CCRT-HSB-2, KE-37, SKW-3, HUT-78, HUT-102, H9, U937, THP-1, HEL, JK-1, CMK, KO-812, MEG-01, etc.); preferably, tissues derived from human brains or human brain regions (e.g., hippocampus) are employed. These tissues may be those derived from normal volunteers or from patients (e.g., with Alzheimer's disease).

The vector to be used for the library may be any of bacteriophage, plasmid, is cosmid, phagemid, and the like. In addition, the DNA can be directly amplified by reverse transcriptase polymerase chain reaction (hereinafter abbreviated as RT-PCR) with total RNA or mRNA fraction prepared from the above-described cells or tissues.

Transfection of the DNA library to the cell line where the two recombinant gene constructs described above are transfected may be carried out by methods described in, e.g., Jikken Igaku (Experimental Medicine), extra issue, Gene Transfer and Expression/Analysis (1994) (published by Yodosha Publishing Co.), etc. These methods include physical means (microinjection, electroporation), chemical means (lipofection, calcium phosphate method) and methods using virus vectors including retrovirus, etc.

[Screening Method of DNA that Controls the Production of Aβ]

The DNA that controls the production of Aβ is investigated as follows. First, the DNA library is transfected to the cell line where the two recombinant gene constructs described above are introduced (hereinafter referred to as the parent strain). Thereafter, for example, when a drug resistant gene is used as the selection marker, a cell bearing the DNA to enhance the concentration of the corresponding drug and become resistant in the presence of the drug even at a high concentration is selected. In the case of using, e.g., puromycin as a drug, the parent strain, to which no DNA library is transfected, is previously examined on sensitivity of the parent strain to puromycin, whereby the puromycin concentration where the parent strain can hardly survive is chosen. For example, puromycin is used in a concentration of 0.1 to 25 μg/ml, preferably 1 to 25 μg/ml, more preferably 5 to 25 μg/ml. Where the reporter gene described above is used in place of the selection marker, its assay may be made in accordance with methods publicly known (e.g., Molecular Biotechnology, 13, 29-43, 1999).

Next, the Aβ produced by the cells selected is assayed, the cell bearing the DNA that increases the production of Aβ is screened, and its DNA is identified by techniques publicly known, such as PCR, etc.

Various methods for assaying Aβ are used, and immunochemical methods using an Aβ-specific antibody are preferred. These methods used include immunoprecipitation, western blotting, enzyme immunoassay and sandwich enzyme immunoassay, or a combination thereof.

As the Aβ-specific antibody, a polyclonal antibody may be used; alternatively, a monoclonal antibody, such as BAN50, BNT77, BS85, BA27, BC05 (Biochemistry, 34, 10272-10278, 1995), 6E10, 4G8 and the like may also be used. In particular, BA27 and BC05 are antibodies selective to Aβ40 and Aβ42/43, respectively. Accordingly, when these antibodies or antibodies having similar selectivity are used, it becomes possible to find a gene that increases the production of Aβ40, a gene that increases the production of Aβ42/43 or a gene that increases the production of-both Aβ40 and Aβ42/43.

Furthermore, since it is considered that the level of secretory APP would indirectly reflect the production of Aβ, the level of secretory APP may be detected by immunochemical methods including immunoprecipitation, western blotting, enzyme immunoassay, sandwich enzyme immunoassay, etc. The candidate genes that increase the production of Aβ thus selected are transfected to other cell line in which βAPP is produced, and Aβ is assayed to verify that the increased Aβ production is actually driven by transfection of these candidate genes. In this case, Aβ62/43 or secretory APP may be likewise used as an indicator. Any cell line may be used as these cell lines for verification, so long as the cell line produces βAPP and has the γ-secretase activity. For example, cell lines such as IMR-32, PC12h, Neuro-2a, SK-N-SH, etc., or cell lines such as HEK-293 introduced with βAPP and PS-1/2, etc. are used. Where the reporter gene above is used as the selection marker in the screening method of the present invention described above, a gene that promotes or inhibit the production of Aβ can be screened by using the reporter activity as an indicator.

[DNA Obtained by Screening and its Products]

The DNA obtained by the screening method of the present invention (hereinafter sometimes simply referred to as the DNA of the present invention) is a DNA that controls the production of Aβ, and may be any one of cDNA, chromosomal DNA and synthetic DNA. Human cDNA and human chromosomal DNA are particularly preferred.

The DNA of the present invention may be the DNA itself obtained by the screening method of the present invention, or may be cDNA or chromosomal DNA, which is obtained by using the DNA or a part thereof as a probe or a primer and cloning in a conventional genetic engineering manner.

The DNA that controls the production of Aβ includes a DNA that increases the production of Aβ.

As the DNA obtained by the screening method of the present invention, the following three genes were specifically acquired.

(1) Human cDNA (accession No. AAH06223) Registered in Genbank

This cDNA has the base sequence represented by SEQ ID NO: 4 and encodes Protein A having the amino acid sequence represented by SEQ ID NO: 1.

This human cDNA showed-extremely high homology to cDNA (accession No. AK003241) registered in Genbank as mouse cDNA encoding Protein A with unknown function. In addition, this cDNA showed high homology also to Syndesmos (Baciu P. C., et al. J. Cell Science 113, 315, 2000; accession No. AF095446) identified as a protein binding to syndecan-4. from chicken in the yeast two-hybrid test.

(2) Herp (Kokame K. et al. J. Biol. Chem. 275: 3286, 2000; Accession No. AB034989) Registered in Genbank

This cDNA has the base sequence represented by SEQ ID NO: 5 and encodes Protein B having the amino acid sequence represented by SEQ ID NO: 2.

Protein B encoded by this cDNA is localized in the endoplasmic reticulum and its expression is induced by endoplasmic reticulum stress, but was a protein with unknown function.

(3) cDNA Containing the 1-389 Partial Sequence from the N-terminus of 5-lipoxygenase (accession no. XM 005818) Registered in Genbank

This cDNA has the base sequence represented by SEQ ID NO: 6 and encodes Protein C having the amino acid sequence represented by SEQ ID NO: 3.

Since overexpression of these three human cDNAs causes the increased production of Aβ, it became clear that these cDNAs are DNAs associated with Alzheimer's disease. Concerning these DNAs, not only the exon region but also the promoter region and the intron region are all critical, in association with Alzheimer's disease.

The peptide, protein or salts thereof, encoded by the DNA obtained by the screening method of the present invention may be any protein derived from any cell or tissue of human or other warm-blooded animals described above, or may be a recombinant protein or a synthetic protein.

The protein of the present invention is a protein containing the same or substantially the same amino acid sequence as the amino acid sequence represented by SEQ ID NO: 1, SEQ ID NO: 2, SEQ ID NO: 3 or SEQ ID NO: 22. Substantially the same amino acid sequence includes an amino acid sequence having at least about 50%, preferably at least about 70% homology, more preferably at least about 80% homology, much more preferably at least about 90% homology, and most preferably at least about 95% homology, to the amino acid sequence represented by, e.g., SEQ ID NO: 1, SEQ ID NO: 2, SEQ ID NO: 3 or SEQ ID NO: 22; etc. Furthermore, the protein of the present invention is preferably a protein containing the same or substantially the same amino acid sequence as the amino acid sequence represented by SEQ ID NO: 1, SEQ ID NO: 2, SEQ ID NO: 3 or SEQ ID NO: 22 and having an activity substantially equivalent to that of the protein containing the amino acid sequence represented by SEQ ID NO: 1, SEQ ID NO: 2, SEQ ID NO: 3 or SEQ ID NO: 22; etc. In terms of the substantially equivalent activity, it is sufficient for the protein to confer the activity of substantially the same quality as that of the protein represented by SEQ ID NO: 1, SEQ ID NO: 2, SEQ ID NO: 3 or SEQ ID NO: 22, and there is no problem if the other activities are different.

As the protein of the present invention, there may be used proteins containing (1) an amino acid sequence represented by SEQ ID NO: 1, SEQ ID NO: 2, SEQ ID NO: 3 or SEQ ID NO: 22, of which at least 1 or 2 (preferably approximately 1 to 30, more preferably approximately 1 to 10 and most preferably several (1 to 5)) amino acids are deleted; (2) an amino acid sequence represented by SEQ ID NO: 1, SEQ ID NO: 2, SEQ ID NO: 3 or SEQ ID NO: 22, to which at least 1 or 2 (preferably approximately 1 to 30, more preferably approximately 1 to 10 and most preferably several (1 to 5)) amino acids are added; (3) an amino acid sequence represented by SEQ ID NO: 1, SEQ ID NO: 2, SEQ ID NO: 3 or SEQ ID NO: 22, in which at least 1 or 2 (preferably approximately 1 to 30, more preferably approximately 1 to 10 and most preferably several (1 to 5)) amino acids are substituted by other amino acids; and (4) an amino acid sequence containing a combination of the above amino acid sequences.

Throughout the present specification, the proteins are represented in accordance with the conventional way of describing peptides, that is, the N-terminus (amino terminus) at the left hand and the C-terminus (carboxyl terminus) at the right hand. In the proteins of the invention including the protein containing the amino acid sequence shown by SEQ ID NO: 1, SEQ ID NO: 2, SEQ ID NO: 3 or SEQ ID NO: 22, the C-terminus may be in any form of a carboxyl group (—COOH), a carboxylate (—COO—), an amide (—CONH₂) or an ester (—COOR).

Examples of the ester group shown by R include a C₁₋₆ alkyl group such as methyl, ethyl, n-propyl, isopropyl, n-butyl, etc.; a C₃₋₈ cycloalkyl group such as cyclopentyl, cyclohexyl, etc.; a C₆₋₁₂ aryl group such as phenyl, α-naphthyl, etc.; a C₇₋₁₄ aralkyl such as a phenyl-C₁₋₂ alkyl group, e.g., benzyl, phenethyl, etc.; an α-naphthyl-C₁₋₂ alkyl group such as α-naphthylmethyl, etc.; and the like. In addition, pivaloyloxymethyl or the like which is used widely as an ester for oral administration may also be used.

Where the protein of the present invention contains a carboxyl group (or a carboxylate) at a position other than the C-terminus, it may be amidated or esterified and such an amide or ester is also included within the protein of the invention. The ester group may be the same group as that described with respect to the above C-terminal group.

Furthermore, examples of the proteins of the invention include variants of the above proteins, wherein the amino group at the N-terminal methionine residue is protected with a protecting group (e.g., a C₁₋₆ acyl group such as a C₂₋₆ alkanoyl group, e.g., formyl group, acetyl group, etc.); those wherein the N-terminal region is cleaved in vivo and the glutamyl group thus formed is pyroglutaminated; those wherein a substituent (e.g., —OH, —SH, amino group, imidazole group, indole group, guanidino group, etc.) on the side chain of an amino acid in the molecule is protected with a suitable protecting group (e.g., a C₁₋₆ acyl group such as a C₂₋₆ alkanoyl group, e.g., formyl group, acetyl group, etc.), or conjugated proteins such as glycoproteins having sugar chains.

Any peptide is usable as the partial peptide of the protein of the present invention (hereinafter sometimes merely referred to as the partial peptide), as long as it has substantially the same amino acid sequence as in the partial peptide of the protein of the present invention described above. Turning to the number of amino acids in the partial peptide of the present invention, preferred peptides are sequences of at least 5, preferably at least 20, and more preferably 100 amino acids in the constituent amino acid sequence represented by, e.g., SEQ ID NO: 1, SEQ ID NO: 2 or SEQ ID NO: 3 described above.

Substantially the same amino acid sequence includes an amino acid sequence having at least about 50% homology, preferably at least about 70% homology, more preferably at least about 80% homology, much more preferably at least about 90% homology, and most preferably at least about 95% homology, to these amino acid sequences.

In the partial peptide of the present invention, at least 1 or 2 (preferably approximately 1 to 10 and more preferably several (1 to 5)) amino acids may be deleted of the above-described amino acid sequence; at least 1 or 2 (preferably approximately 1 to 10 and more preferably several (1 to 5)) amino acids may be added to the amino acid sequence; or, at least 1 or 2 (preferably approximately 1 to 10 and more preferably several (1 to 5)) amino acids in the amino acid sequence may be substituted by other amino acids.

In the partial peptide of the invention, the C-terminus may be in any form of a carboxyl group (—COOH), a carboxylate (—COO⁻), an amide (—CONH₂) or an ester (—COOR). Herein, R in the ester has the same significance as defined above.

Furthermore, examples of the partial peptides of the invention include, as in the proteins of the present invention described above, those wherein the amino group at the N-terminal methionine residue is protected with a protecting group; those wherein the N-terminal region is cleaved in vivo and the glutamyl group thus formed is pyroglutaminated; those wherein a substituent on the side chain of an amino acid in the molecule is protected with a suitable protecting group, or conjugated peptides having sugar chains, so-called glycopeptides.

The salts of the proteins or partial peptides of the present invention include physiologically acceptable salts with acids or bases, preferably physiologically acceptable acid addition salts. As such salts, there are used salts with, for example, inorganic acids (e.g., hydrochloric acid, phosphoric acid, hydrobromic acid, sulfuric acid), or with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid), etc.

The peptide or protein encoded by the DNA obtained by the screening method of the present invention, including the protein containing the amino acid sequence represented by SEQ ID NO: 1, SEQ ID NO: 2, SEQ ID NO: 3 or SEQ ID NO: 22 (hereinafter merely referred to as the protein, including the peptide), or its salts may be manufactured from the aforesaid cells or tissues of human or other warm-blooded animals by applying publicly known methods for purification of peptides of proteins, or may be manufactured by culturing a transformant bearing a DNA encoding the protein of the present invention, which will be described hereinafter. Furthermore, the protein or its salts may be manufactured by the protein synthesis or its modifications, which will be also described-later.

Where the protein or its salts are produced from the tissues or cells of human or other warm-blooded animals, the tissues or cells of human or other warm-blooded animals are homogenized, then extracted with an acid or the like, and the extract is isolated and purified by a combination of chromatography techniques such as reverse phase chromatography, ion exchange chromatography, and the like.

To synthesize the protein of the present invention, its partial peptide, or salts or amides thereof, commercially available resins that are used for protein synthesis may be used. Examples of such resins include chloromethyl resin, hydroxymethyl resin, benzhydrylamine resin, aminomethyl resin, 4-benzyloxybenzyl alcohol resin, 4-methylbenzhydrylamine resin, PAM resin, 4-hydroxymethylmehtylphenyl acetamidomethyl resin, polyacrylamide resin, 4-(2′,4′-dimethoxyphenyl-hydroxymethyl)phenoxy resin, 4-(2′,4′-dimethoxyphenyl-Fmoc-aminoethyl) phenoxy resin, etc. Using these resins, amino acids in which α-amino groups and functional groups on the side chains are appropriately protected are condensed on the resin in the order of the sequence of the objective protein according to various condensation methods publicly known in the art. At the end of the reaction, the protein is excised from the resin and at the same time, the protecting groups are removed. Then, intramolecular disulfide bond-forming reaction is performed in a highly diluted solution to obtain the objective protein, partial peptide or amides thereof.

For condensation of the protected amino acids described above, a variety of activation reagents for protein synthesis may be used, but carbodiimides are particularly preferably employed. Examples of such carbodiimides include DCC, N,N′-diisopropylcarbodiimide, N-ethyl-N′-(3-dimethylaminopropyl)carbodiimide, etc. For activation by these reagents, the protected amino acids in combination with a racemization inhibitor (e.g., HOBt, HOOBt) are added directly to the resin, or the protected amino acids are previously activated in the form of symmetric acid anhydrides, HOBt esters or HOOBt esters, followed by adding the thus activated protected amino acids to the resin.

Solvents suitable for use to activate the protected amino acids or condense with the resin may be chosen from solvents that are known to be usable for protein condensation reactions. Examples of such solvents are acid amides such as N,N-dimethylformamide, N,N-dimethylacetamide, N-methylpyrrolidone; halogenated hydrocarbon such as methylene chloride, chloroform; alcohols such as trifluoroethanol; sulfoxides such as dimethylsulfoxide; ethers such as pyridine, dioxan, tetrahydrofuran; nitriles such as acetonitrile, propionitrile; esters such as methyl acetate, ethyl acetate, or appropriate mixtures of these solvents. The reaction temperature is appropriately chosen from the range known to be applicable to protein bond-forming reactions and is usually selected in the range of approximately −20° C. to 50° C. The activated amino acid derivatives are used generally in an excess of 1.5 to 4 times. The condensation is examined using the ninhydrin reaction; when the condensation is insufficient, the condensation can be completed by repeating the condensation reaction without removal of the protecting groups. When the condensation is yet insufficient even after repeating the reaction, unreacted amino acids are acetylated with acetic anhydride or acetylimidazole to cancel any possible adverse affect on the subsequent reaction.

Examples of the protecting groups used to protect amino groups of the starting materials include Z, Boc, tertiary-pentyloxycarbonyl, isobornyloxycarbonyl, 4-methoxybenzyloxycarbonyl, Cl-Z, Br-Z, adamantyloxycarbonyl, trifluoroacetyl, phthaloyl, formyl, 2-nitrophenylsulphenyl, diphenylphosphinothioyl, Fmoc, etc.

A carboxyl group can be protected by converting the carboxyl group into, e.g., alkyl esters (e.g., straight-chain, branched or cyclic alkyl esters such as methyl, ethyl, propyl, butyl, tertiary-butyl, cyclopentyl, cyclohexyl, cycloheptyl, cyclooctyl or 2-adamantyl ester, etc.), aralkyl esters (e.g., benzyl ester, 4-nitrobenzyl ester, 4-methoxybenzyl ester, 4-chlorobenzyl ester, benzhydryl ester), phenacine esters, benzyloxycarbonyl hydrazide, tertiary-butoxycarbonyl hydrazide, trityl hydrazide, or the like.

The hydroxyl group of serine can be protected by, for example, its esterification or etherification. Examples of groups appropriately used for the esterification include a lower alkanoyl group such as acetyl group, etc., an aroyl group such as benzoyl group, etc., and a group derived from carbon such as benzyloxycarbonyl group and ethoxycarbonyl group. Examples of the group appropriately used for the etherification include benzyl group, tetrahydropyranyl group, t-butyl group, and the like.

Examples of groups for protecting the phenolic hydroxyl group of tyrosine include Bzl, Cl₂-Bzl, 2-nitrobenzyl, Br-Z, tertiary-butyl, etc.

Examples of groups used to protect the imidazole moiety of histidine include Tos, 4-methoxy-2,3,6-trimethylbenzenesulfonyl, DNP, benzyloxymethyl, Bum, Boc, Trt, Fmoc, etc.

Examples of the activated carboxyl groups in the starting material include the corresponding acid anhydrides, azides, activated esters (esters with alcohols (e.g., pentachlorophenol, 2,4,5-trichlorophenol, 2,4-dinitrophenol, cyanomethyl alcohol, p-nitrophenol, HONB, N-hydroxysuccimide, N-hydroxyphthalimide, HOBt)). As the activated form of the amino groups in the starting material, the corresponding phosphoric amides are employed.

To eliminate (split off) the protecting groups, there are employed catalytic reduction in a hydrogen gas flow in the presence of a catalyst such as Pd-black or Pd-carbon; an acid treatment with anhydrous hydrogen fluoride, methanesulfonic acid, trifluoromethanesulfonic acid or trifluoroacetic acid, or a mixture solution of these acids; a treatment with a base such as diisopropylethylamine, triethylamine, piperidine or piperazine; and reduction with sodium in liquid ammonia. The elimination of the protecting group by the acid treatment described above is carried out generally at a temperature of approximately −20° C. to 40° C. In the acid treatment, it is efficient to add a cation scavenger such as anisole, phenol, thioanisole, m-cresol, p-cresol, dimethylsulfide, 1,4-butanedithiol or 1,2-ethanedithiol. Furthermore, 2,4-dinitrophenyl group known as the protecting group for the imidazole of histidine is removed by a treatment with thiophenol. Formyl group used as the protecting group of the indole of tryptophan is eliminated by the aforesaid acid treatment in the presence of 1,2-ethanedithiol, 1,4-butanedithiol or the like, as well as by a treatment with an alkali such as a diluted sodium hydroxide solution and dilute ammonia.

Protection of functional groups that should not be involved in the reaction of the starting materials, protecting groups, elimination of the protecting groups and activation of functional groups involved in the reaction may be appropriately selected from publicly known groups and publicly known means.

In another method for obtaining the amides of the protein, for example, the α-carboxyl group of the carboxy terminal amino acid is first protected by amidation; the peptide (protein) chain is then extended from the amino group side to a desired length. Thereafter, a protein in which only the protecting group of the N-terminal a-amino group has been eliminated from the peptide chain and a protein in which only the protecting group of the C-terminal carboxyl group has been eliminated are manufactured. The two proteins are condensed in a mixture of the solvents described above. The details of the condensation reaction are the same as described hereinabove. After the protected protein obtained by the condensation is purified, all the protecting groups are eliminated by the method described above to give the desired crude protein. This crude protein is purified by various known purification means. Lyophilization of the major fraction gives the amide of the desired protein.

To prepare the esterified protein, for example, the α-carboxyl group of the carboxy terminal amino acid is condensed with a desired alcohol to prepare the amino acid ester, which is followed by procedure similar to the preparation of the amidated protein above to give the desired esterified protein.

The partial peptide or salts of the present invention can be manufactured by publicly known methods for peptide synthesis, or by cleaving the protein of the present invention with an appropriate peptidase. For the methods for peptide synthesis, for example, either solid phase synthesis or liquid phase synthesis may be used. That is, the partial peptide or amino acids that can construct the protein of the present invention are condensed with the remaining part of the partial peptide of the present invention. Where the product contains protecting groups, these protecting groups are removed to give the desired peptide. Publicly known methods for condensation and elimination of the protecting groups are described in 1)-5) below.

1) M. Bodanszky & M. A. Ondetti: Peptide Synthesis, Interscience Publishers, New York (1966)

2) Schroeder & Luebke: The Peptide, Academic Press, New York (1965)

3) Nobuo Izumiya, et al.: Peptide Gosei-no-Kiso to Jikken (Basics and experiments of peptide synthesis), published by Maruzen Co. (1975)

4) Haruali Yajima & Shunpei Sakakibara: Seikagaku Jikken Koza (Biochemical Experiment) 1, Tanpakushitsu no Kagaku (Chemistry of Proteins) IV, 205 (1977)

5) Haruaki Yajima ed.: Zoku Iyakuhin no Kaihatsu (A sequel to Development of Pharmaceuticals), Vol. 14, Peptide Synthesis, published by Hirokawa Shoten

After completion of the reaction, the partial peptide of the present invention can be purified and isolated by a combination of conventional purification methods such as solvent extraction, distillation, column chromatography, liquid chromatography and recrystallization to give the partial peptide of the present invention. When the partial peptide obtained by the above methods is in a free form, the partial peptide can be converted into an appropriate salt by a publicly known method; conversely when the partial peptide is obtained in a salt form, it can be converted into a free form by a publicly known method.

The polynucleotide coding for the protein encoded by the DNA obtained by the screening method of the present invention may be prepared in a manner similar to the polynucleotide coding for the protein containing the amino acid sequence represented by SEQ ID NO: 1, SEQ ID NO: 2, SEQ ID NO: 3 or SEQ ID NO: 22, which will be explained below.

The polynucleotide coding for the protein containing the amino acid sequence represented by SEQ ID NO: 1, SEQ ID NO: 2, SEQ ID NO: 3 or SEQ ID NO: 22 may be any polynucleotide so long as it contains the base sequence (DNA or RNA, preferably DNA) encoding the protein having the amino acid sequence represented by SEQ ID NO: 1, SEQ ID NO: 2, SEQ ID NO: 3 or SEQ ID NO: 22. The said polynucleotide is a DNA or RNA such as mRNA, etc. encoding the protein having the amino acid sequence represented by SEQ ID NO: 1, SEQ ID NO: 2, SEQ ID NO: 3 or SEQ ID NO: 22, which may be either e double-stranded or single-stranded. Where the polynucleotide is double-stranded, it may be double-stranded DNA, double-stranded RNA or DNA:RNA hybrid. Where the polynucleotide is single-stranded, it may be a sense strand (i.e., a coding strand) or an antisense strand (i.e., a non-coding strand).

The DNA encoding the protein containing the amino acid sequence represented by SEQ ID NO: 1, SEQ ID NO: 2, SEQ ID NO: 3 or SEQ ID NO: 22 may be any one of genomic DNA, genomic DNA library, cDNA derived from the cells/tissues described above, cDNA library derived from the cells/tissues described above and synthetic DNA. The vector to be used for the library may be any of bacteriophage, plasmid, cosmid, phagemid and the like. In addition, the DNA can be directly amplified by reverse transcriptase polymerase chain reaction (hereinafter abbreviated as RT-PCR) using the total RNA or mRNA fraction prepared from the above-described cells/tissues.

Specifically, any DNA is usable as the DNA encoding the protein of the present invention containing the amino acid sequence represented by SEQ ID NO: 1, SEQ ID NO: 2, SEQ ID NO: 3 or SEQ ID NO: 22, so long as the DNA is, for example, a DNA containing the base sequence represented by SEQ ID NO: 4, SEQ ID NO: 5, SEQ ID NO: 6 or SEQ ID NO: 21, or a DNA hybridizable to a DNA having the base sequence represented by SEQ ID NO: 4, SEQ ID NO: 5, SEQ ID NO: 6 or SEQ ID NO: 21 under high stringent conditions and encoding a protein having an activity of increasing the production of Aβ, which is substantially equivalent to that of the protein containing the amino acid sequence represented by SEQ ID NO: 1, SEQ ID NO: 2, SEQ ID NO: 3 or SEQ ID NO: 22.

Examples of the DNA that is hybridizable to the DNA having the base sequence represented by SEQ ID NO: 4, SEQ ID NO: 5, SEQ ID NO: 6 or SEQ ID NO: 21 under high stringent conditions include a DNA having at least about 70% homology, preferably at least about 80% homology, more preferably at least about 90% homology, and most preferably at least about 95% homology, to the base sequence represented by SEQ ID NO: 4, SEQ ID NO: 5, SEQ ID NO: 6 or SEQ ID NO: 21.

The hybridization can be carried out by publicly known methods or by a modification thereof, for example, according to the method described in Molecular Cloning, 2nd Ed. (J. Sambrook et al., Cold Spring Harbor Lab. Press, (1989). A commercially available library may also be used according to the instructions of the attached manufacturer's protocol. More preferably, the hybridization can be carried out under high stringent conditions.

The high stringent conditions used herein are, for example, those in a sodium concentration at about 19 mM to about 40 mM, preferably about 19 mM to about 20 mM at a temperature of approximately 50 to 70° C., preferably approximately 60 to 65° C. In particular, hybridization conditions in a sodium concentration at about 19 mM at a temperature of about 65° C. are most preferred.

The polynucleotide comprising a part of the base sequence of the DNA encoding the protein of the present invention having the amino acid sequence represented by SEQ ID NO: 1, SEQ ID NO: 2, SEQ ID NO: 3 or SEQ ID NO: 22, or a part of the base sequence complementary to the DNA, is used to mean to embrace not only the DNA encoding the partial peptide of the present invention described below but also RNA.

Using the polynucleotide coding for the protein of the present invention containing the amino acid sequence represented by SEQ ID NO: 1, SEQ ID NO: 2, SEQ ID NO: 3 or SEQ ID NO: 22, mRNA for the protein of the present invention containing the amino acid sequence represented by SEQ ID NO: 1, SEQ ID NO: 2 or SEQ ID NO: 3 can be quantified by, e.g., a publicly known method described in Jikken Igaku (Experimental Medicine), extra issue, “New PCR and its application,” 15 (7), 1997, or modifications thereof.

According to the present invention, antisense polynucleotides (nucleic acids) that can inhibit the replication or expression of protein genes containing the amino acid sequence represented by SEQ ID NO: 1, SEQ ID NO: 2, SEQ ID NO: 3 or SEQ ID NO: 22 can be designed and synthesized based on the base sequence information of the cloned or determined DNA encoding the protein containing the amino acid sequence represented by SEQ ID NO: 1, SEQ ID NO: 2, SEQ ID NO: 3 or SEQ ID NO: 22. Such a polynucleotide (nucleic acid) is capable of hybridizing to RNA of a gene for the protein containing the amino acid sequence represented by SEQ ID NO: 1, SEQ ID NO: 2, SEQ ID NO: 3 or SEQ ID NO: 22, thereby enabling to inhibit the synthesis or function of said RNA, or enabling to modulate/control the expression of a gene for the protein containing the amino acid sequence represented by SEQ ID NO: 1, SEQ ID NO: 2, SEQ ID NO: 3 or SEQ ID NO: 22 via interaction with the RNA associated with the protein containing the amino acid sequence represented by SEQ ID NO: 1, SEQ ID NO: 2, SEQ ID NO: 3 or SEQ ID NO: 22. Polynucleotides complementary to the selected sequences of RNA associated with the protein containing the amino acid sequence represented by SEQ ID NO: 1, SEQ ID NO: 2, SEQ ID NO: 3 or SEQ ID NO: 22 and polynucleotides specifically hybridizable to the RNA associated the protein containing the amino acid sequence represented by SEQ ID NO: 1, SEQ ID NO: 2, SEQ ID NO: 3 or SEQ ID NO: 22 are useful in modulating/controlling the expression of a gene for the protein containing the amino acid sequence represented by SEQ ID NO: 1, SEQ ID NO: 2, SEQ ID NO: 3 or SEQ ID NO: 22 in vivo and in vitro, and useful for the treatment or diagnosis of diseases, etc. In these polynucleotides, the 5′ end hairpin loop, 5′ end 6-base-pair repeats, 5′ end untranslated region, polypeptide translation initiation codon, protein coding region, ORF translation termination codon, 3′ end untranslated region, 3′ end palindrome region and 3′ end hairpin loop of the protein gene containing the amino acid sequence represented by SEQ ID NO: 1, SEQ ID NO: 2, SEQ ID NO: 3 or SEQ ID NO: 22, may be selected and prepared as preferred target regions, though any other region may also be selected as a target in the protein genes containing the amino acid sequence represented by SEQ ID NO: 1, SEQ ID NO: 2, SEQ ID NO: 3 or SEQ ID NO: 22.

The relationship between the targeted nucleic acids and the polynucleotides complementary to at least a part of the target, or the relationship between the target and the polynucleotides hybridizable to the target, can be denoted to be “antisense.” Examples of the antisense polynucleotides include polydeoxynucleotides containing 2-deoxy-D-ribose, polynucleotides containing D-ribose, any other type of polynucleotides which are N-glycosides of a purine or pyrimidine base, or other polymers containing non-nucleotide backbones (e.g., protein, nucleic acids and synthetic sequence-specific nucleic acid polymers commercially available) or other polymers containing particular linkages (provided that the polymers contain nucleotides having such a configuration that allows base pairing or base stacking, as is found in DNA or RNA), etc. The antisense polynucleotides may be double-stranded DNA, single-stranded DNA, single-stranded RNA or a DNA:RNA hybrid, and may further include unmodified polynucleotides (or unmodified oligonucleotides), those with publicly known types of modifications, for example, those with labels known in the art, those with caps, methylated polynucleotides, those with substitution of one or more naturally occurring nucleotides by their analog, those with intramolecular modifications of nucleotides such as those with uncharged linkages (e.g., methyl phosphonates, phosphotriesters, phosphoramidates, carbamates, etc.) and those with charged linkages or sulfur-containing linkages (e.g., phosphorothioates, phosphorodithioates, etc.), those having side chain groups such as proteins (nucleases, nuclease inhibitors, toxins, antibodies, signal peptides, poly-L-lysine, etc.), saccharides (e.g., monosaccharides, etc.), those with intercalators (e.g., acridine, psoralen, etc.), those containing chelators (e.g., metals, radioactive metals, boron, oxidative metals, etc.), those containing alkylating agents, those with modified linkages (e.g., a anomeric nucleic acids, etc.), and the like. Herein the terms “nucleoside”, “nucleotide” and “nucleic acid” are used to refer to moieties that contain not only the purine and pyrimidine bases, but also other heterocyclic bases, which have been modified. Such modified products may include methylated purines and pyrimidines, acylated purines and pyrimidines and other heterocyclic rings. Modified nucleotides and modified nucleotides also include modifications on the sugar moiety, wherein, for example, one or more hydroxyl groups may optionally be substituted with a halogen atom(s), an aliphatic group(s), etc., or may be converted into the corresponding functional groups such as ethers, amines, or the like.

The antisense polynucleotide (nucleic acid) of the present invention is RNA, DNA or a modified nucleic acid (RNA, DNA). Specific examples of the modified nucleic acid are, but not limited to, sulfur and thiophosphate derivatives of nucleic acids and those resistant to degradation of polynucleoside amides or oligonucleoside amides. The antisense nucleic acids of the present invention can be modified preferably based on the following design, that is, by increasing the intracellular stability of the antisense nucleic acid, increasing the cell permeability of the antisense nucleic acid, increasing the affinity of the nucleic acid to the targeted sense strand to a higher level, or minimizing the toxicity, if any, of the antisense nucleic acid.

Many of such modifications are known in the art, as disclosed in J. Kawakami, et al., Pharm. Tech. Japan, Vol. 8, pp. 247, 1992; Vol. 8, pp. 395, 1992; S. T. Crooke, et al. ed., Antisense Research and Applications, CRC Press, 1993; etc.

The antisense nucleic acid of the present invention may contain altered or modified sugars, bases or linkages. The antisense nucleic acid may also be provided in a particular form such as liposomes, microspheres, or may be applied to gene therapy, or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase uptake of the nucleic acid. Preferred examples of the lipids to be attached are cholesterols or derivatives thereof (e.g., cholesteryl chloroformate, cholic acid, etc.). These moieties may be attached to the nucleic acid at the 3′ or 5′ ends thereof and may also be attached thereto through a base, sugar, or intramolecular nucleoside linkage. Other moieties may be capping groups specifically placed at the 3′ or 5′ ends of the nucleic acid to prevent degradation by nucleases such as exonuclease, RNase, etc. Such capping groups include, but are not limited to, hydroxyl protecting groups known in the art, including glycols such as polyethylene glycol, tetraethylene glycol and the like.

The inhibitory activity of the antisense nucleic acid can be examined using the transformant of the present invention, the gene expression system of the present invention in vivo or in vitro, or the translation system of the protein containing the amino acid sequence represented by SEQ ID NO: 1, SEQ ID NO: 2, SEQ ID NO: 3 or SEQ ID NO: 22 in vivo and in vitro. The nucleic acid can be applied to cells by a variety of publicly known methods.

The DNA encoding the partial peptide of the present invention may be any DNA, so long as it contains the base sequence encoding the partial peptide of the present invention described above. The DNA may also be any of genomic DNA, genomic DNA library, cDNA derived from the cells and tissues described above, cDNA library derived from the cells and tissues described above and synthetic DNA. The vector to be used for the library may be any of bacteriophage, plasmid, cosmid and phagemid. The DNA may also be directly amplified by reverse transcriptase polymerase chain reaction (hereinafter abbreviated as RT-PCR) using the mRNA fraction prepared from the cells and tissues described above.

Specifically, the DNA encoding the partial peptide of the present invention may be any one of, for example, (1) a DNA containing a partial base sequence of the DNA having the base sequence represented by SEQ ID NO: 4, SEQ ID NO: 5, SEQ ID NO: 6 or SEQ ID NO: 21, or (2) a DNA having a base sequence hybridizable to the base sequence represented by SEQ ID NO: 4, SEQ ID NO: 5, SEQ ID NO: 6 or SEQ ID NO: 21 under high stringent conditions and having a partial base sequence of a DNA encoding a protein which has an activity of increasing the production of Aβ substantially equivalent to that of the protein having the amino acid sequence represented by SEQ ID NO: 1, SEQ ID NO: 2, SEQ ID NO: 3 or SEQ ID NO: 22; etc.

Specific examples of the DNA that is hybridizable to the partial base sequence represented by SEQ ID NO: 4, SEQ ID NO: 5, SEQ ID NO: 6 or SEQ ID NO: 21 include DNA containing a base sequence having at least about 70% homology, preferably at least about. 80% homology, more preferably at least about 90% homology and further more preferably at least about 95% homology, to the base sequence represented by SEQ ID NO: 4, SEQ ID NO: 5, SEQ ID NO: 6 or SEQ ID NO: 21.

For cloning of the DNA that completely encodes the protein of the present invention containing the amino acid sequence represented by SEQ ID NO: 1, SEQ ID NO: 2, SEQ ID NO: 3 or SEQ ID NO: 22 or its partial peptide (hereinafter sometimes collectively referred to as the protein of the present invention), the DNA may be either amplified by PCR using synthetic DNA primers having a part of the base sequence encoding the protein of the present invention, or the DNA inserted into an appropriate vector may be selected by hybridization with a labeled DNA fragment or synthetic DNA that encodes a part or entire region of the protein of the present invention. The hybridization can be carried out, for example, according to the method described in Molecular Cloning, 2nd (J. Sambrook et al., Cold Spring Harbor Lab. Press, 1989), etc. The hybridization may also be performed using commercially available library in accordance with the protocol described in the attached instructions.

Substitution of the base sequence in the DNA may be effected by publicly known methods such as the Gapped duplex method or the Kunkel method, or modifications thereof, by using publicly known kits available as, e.g., Mutan™-G or Mutan™-K (both manufactured by Takara Shuzo Co., Ltd.), etc.

The cloned DNA encoding the protein of the present invention can be used as it is, depending upon purpose or, if desired, after digestion with a restriction enzyme or after addition of a linker thereto. The DNA may contain ATG as a translation initiation codon at the 5′ end thereof and may further contain TAA, TGA or TAG as a translation termination codon at the 3′ end thereof. These translation initiation and termination codons may also be added by using an appropriate synthetic DNA adapter.

The expression vector for the protein of the present invention can be manufactured, for example, by (a) excising the desired DNA fragment from the DNA (e.g., cDNA) encoding the protein of the present invention, and then (b) ligating the DNA fragment with an appropriate expression vector downstream a promoter in the vector.

Examples of the vector include plasmids derived form E. coli (e.g., pBR322, pBR325, pUC₁₂, pUC₁₃), plasmids derived from Bacillus subtilis (e.g., pUB110, pTP5, pC₁₉₄), plasmids derived from yeast (e.g., pSH19, pSH15), bacteriophages such as λ phage, etc., animal viruses such as retrovirus, vaccinia virus, baculovirus, etc. as well as pA1-11, pXT1, pRc/CMV, pRc/RSV, pcDNAI/Neo, etc.

The promoter used in the present invention may be any promoter if it matches well with a host to be used for gene expression. In the case of using animal cells as the host, examples of the promoter include SRα promoter, SV40 promoter, LTR promoter, CMV promoter, HSV-TK promoter, etc.

Among them, CMV promoter or SRα promoter is preferably used. Where the host is bacteria of the genus Escherichia, preferred examples of the promoter include trp promoter, lac promoter, recA promoter, λP_{L} promoter, 1pp promoter, etc. In the case of using bacteria of the genus Bacillus as the host, preferred example of the promoter are SPO1 promoter, SPO2 promoter, penP promoter, etc. When yeast is used as the host, preferred examples of the promoter are PHO5 promoter, PGK promoter, GAP promoter, ADH promoter, etc. When insect cells are used as the host, preferred examples of the promoter include polyhedrin prompter, P10 promoter, etc.

In addition to the foregoing examples, the expression vector may further optionally contain an enhancer, a splicing signal, a polyA addition signal, a selection marker, SV40 replication origin (hereinafter sometimes abbreviated as SV40ori), etc. Examples of the selection marker include dihydrofolate reductase (hereinafter sometimes abbreviated as dhfr) gene [methotrexate (MTX) resistance], ampicillin resistant gene (hereinafter sometimes abbreviated as Amp^{r}), neomycin resistant gene (hereinafter sometimes abbreviated as Neo^{r}, G418 resistance), etc. In particular, when dhfr gene is used as the selection marker in CHO (dhfr⁻) cells, selection can also be made on thymidine free media.

If necessary, a signal sequence that matches with a host is added to the N-terminus of the protein of the present invention. Examples of the signal sequence that can be used are PhoA signal sequence, OmpA signal sequence, etc. in the case of using bacteria of the genus Escherichia as the host; α-amylase signal sequence, subtilisin signal sequence, etc. in the case of using bacteria of the genus Bacillus as the host; MFα signal sequence, SUC₂ signal sequence, etc. in the case of using yeast as the host; and insulin signal sequence, α-interferon signal sequence, antibody molecule signal sequence, etc. in the case of using animal cells as the host, respectively.

Using the vector containing the DNA encoding the protein of the present invention thus constructed, transformants can be manufactured.

Examples of the host, which may be employed, are bacteria belonging to the genus Escherichia, bacteria belonging to the genus Bacillus, yeast, insect cells, insects and animal cells, etc.

Specific examples of the bacteria belonging to the genus Escherichia include Escherichia coli K12 DH1 [Proc. Natl. Acad. Sci. U.S.A., 60, 160 (1968)], JM103 [Nucleic Acids Research, 9, 309 (1981)], JA221 [Journal of Molecular Biology, 120, 517 (1978)], HB101 [Journal of Molecular Biology, 41, 459 (1969)], C600 [Genetics, 39, 440 (1954)], etc.

Examples of the bacteria belonging to the genus Bacillus include Bacillus subtilis MI114 [Gene, 24, 255 (1983)], 207-21 [Journal of Biochemistry, 95, 87 (1984)], etc.

Examples of yeast include Saccharomyces cereviseae AH22, AH22R⁻, NA87-11A, DKD-5D, 20B-12, Schizosaccharomyces pombe NCYC1913, NCYC2036, Pichia pastoris, etc.

Examples of insect cells include, for the virus AcNPV, Spodoptera frugiperda cells (Sf cells), MG1 cells derived from mid-intestine of Trichoplusia ni, High Five™ cells derived from egg of Trichoplusia ni, cells derived from Mamestra brassicae, cells derived from Estigmena acrea, etc.; and for the virus BmNPV, Bombyx mori N cells (BmN cells), etc. are used. Examples of the Sf cell which can be used are Sf9 cells (ATCC CRL1711), Sf21 cells (both cells are described in Vaughn, J. L. et al., In Vivo, 13, 213-217 (1977)), etc.

As the insect, for example, a larva of silkworm can be used [Maeda, et al., Nature, 315, 592 (1985)].

Examples of animal cells include monkey cells COS-7, Vero, Chinese hamster cells CHO (hereinafter referred to as CHO cells), dhfr gene deficient Chinese hamster cells CHO (hereinafter simply referred to as CHO(dhfr⁻) cell), mouse L cells, mouse AtT-20, mouse myeloma cells, rat GH3, human FL cells, etc.

Bacteria belonging to the genus Escherichia can be transformed, for example, by the method described in Proc. Natl. Acad. Sci. U.S.A., 69, 2110 (1972), Gene, 17, 107 (1982), etc.

Bacteria belonging to the genus Bacillus can be transformed, for example, by the method described in, e.g., Molecular & General Genetics, 168, 111 (1979), etc.

Yeast can be transformed, for example, by the method described in Methods in Enzymology, 194, 182-187 (1991), Proc. Natl. Acad. Sci. U.S.A., 75, 1929 (1978), etc.

Insect cells or insects can be transformed, for example, according to the method described in Bio/Technology, 6, 47-55 (1988), etc.

Animal cells can be transformed, for example, according to the method described in Saibo Kogaku (Cell Engineering), extra issue 8, Shin Saibo Kogaku Jikken Protocol (New Cell Engineering Experimental Protocol), 263-267 (1995), published by Shujunsha, or Virology, 2, 456 (1973).

Thus, the transformant transformed with the expression vector containing the DNA encoding the protein of the present invention can be obtained.

Where the host is bacteria belonging to the genus Escherichia or the genus Bacillus, the transformant can be appropriately incubated in a liquid medium which contains materials required for growth of the transformant such as carbon sources, nitrogen sources, inorganic materials, and so on. Examples of the carbon sources include glucose, dextrin, soluble starch, sucrose, etc. Examples of the nitrogen sources include inorganic or organic materials such as ammonium salts, nitrate salts, corn steep liquor, peptone, casein, meat extract, soybean cake, potato extract, etc. Examples of the inorganic materials are calcium chloride, sodium dihydrogenphosphate, magnesium chloride, etc. In addition, yeast extract, vitamins, growth promoting factors etc. may also be added to the medium. Preferably, pH of the medium is adjusted to about 5 to about 8.

A preferred example of the medium for incubation of the bacteria belonging to the genus Escherichia is M9 medium supplemented with glucose and Casamino acids (Miller, Journal of Experiments in Molecular Genetics, 431-433, Cold Spring Harbor Laboratory, New York, 1972). If necessary, a chemical such as 3β-indolylacrylic acid can be added to the medium thereby to activate the promoter efficiently. Where the bacteria belonging to the genus Escherichia are used as the host, the transformant is usually cultivated at about 15° C. to about 43° C. for about 3 hours to about 24 hours. If necessary, the culture may be aerated or agitated.

Where the bacteria belonging to the genus Bacillus are used as the host, the transformant is cultivated generally at about 30° C. to about 40° C. for about 6 hours to about 24 hours. If necessary, the culture can be aerated or agitated.

Where yeast is used as the host, the transformant is cultivated, for example, in Burkholder's minimal medium [Bostian, K. L. et al., Proc. Natl. Acad. Sci. U.S.A., 77, 4505 (1980)] or in SD medium supplemented with 0.5% Casamino acids [Bitter, G. A. et al., Proc. Natl. Acad. Sci. U.S.A., 81, 5330 (1984)]. Preferably, pH of the medium is adjusted to about 5 to about 8. In general, the transformant is cultivated at about 20° C. to about 35° C. for about 24 hours to about 72 hours. If necessary, the culture can be aerated or agitated.

Where insect cells or insects are used as the host, the transformant is cultivated in, for example, Grace's Insect Medium (Grace, T. C. C., Nature, 195, 788 (1962)) to which an appropriate additive such as immobilized 10% bovine serum is added. Preferably, pH of the medium is adjusted to about 6.2 to about 6.4. Normally, the transformant is cultivated at about 27° C. for about 3 days to about 5 days and, if necessary, the culture can be aerated or agitated.

Where animal cells are employed as the host, the transformant is cultivated in, for example, MEM medium containing about 5% to about 20% fetal cow serum [Science, 122, 501 (1952)], DMEM medium [virology, 8, 396 (1959)], RPMI 1640 medium [The Journal of the American Medical Association, 199, 519 (1967)], 199 medium [Proceeding of the Society for the Biological Medicine, 73, 1 (1950)], etc. Preferably, pH of the medium is adjusted to about 6 to about 8. The transformant is usually cultivated at about 30° C. to about 40° C. for about 15 hours to about 60 hours and, if necessary, the culture can be aerated or agitated.

As described above, the protein of the present invention can be produced in cell membranes of the transformant.

The protein of the present invention can be separated and purified from the culture described above by the following procedures.

When the protein of the present invention is extracted from the cultured bacterial cells or cells, after cultivation these cells are collected by a publicly known method and suspended in an appropriate buffer. The bacterial cells or cells are then disrupted by publicly known methods such as ultrasonication, a treatment with lysozyme and/or freeze-thaw cycling, followed by centrifugation, filtration, etc. Thus, the crude extract of the protein of the present invention can be obtained. The buffer may contain a protein modifier such as urea or guanidine hydrochloride, or a surfactant such as Triton X-100™, etc. When the protein of the present invention is secreted in the culture solution, after completion of the cultivation the supernatant can be separated from the bacterial cells or cells to collect the supernatant by a publicly known method.

The protein contained in the supernatant or the extract thus obtained can be purified by appropriately combining the publicly known methods for separation/purification. Such publicly known methods for separation/purification include a method utilizing difference in solubility such as salting out, solvent precipitation, etc.; a method utilizing mainly difference in molecular weight such as dialysis, ultrafiltration, gel filtration, SDS-polyacrylamide gel electrophoresis, etc.; a method utilizing difference in electric charge such as ion exchange chromatography, etc.; a method utilizing difference in specific affinity such as affinity chromatography, etc.; a method utilizing difference in hydrophobicity such as reverse phase high performance liquid chromatography, etc.; a method utilizing difference in isoelectric point such as isoelectrofocusing electrophoresis; and the like.

When the protein of the present invention thus obtained is in a free form, it can be converted into the salt by publicly known methods or modifications thereof. On the other hand, when the protein is obtained in the form of a salt, it can be converted into the free form or in the form of a different salt by publicly known methods or modifications thereof.

The protein of the present invention produced by the recombinant can be treated, prior to or after the purification, with an appropriate protein modifying enzyme so that the protein can be appropriately modified and partially removed a polypeptide. Examples of the protein-modifying enzyme include trypsin, chymotrypsin, arginyl endopeptidase, protein kinase, glycosidase or the like.

The activity of the thus produced protein of the present invention or salts thereof can be assayed by a binding test to a labeled ligand, enzyme immunoassay using a specific antibody, an enzyme activity, a signal transduction activity, a substance transport activity, a substance permeability activity, an activity of increasing the production of Aβ, etc.

[Antibody]

The antibody to the protein of the present invention, its partial peptides or salts thereof (hereinafter sometimes collectively referred to as the protein of the present invention) may be any of a polyclonal antibody and a monoclonal antibody, as long as it is capable of recognizing the protein of the present invention, its partial peptides, or salts thereof.

The antibody to the protein of the present invention, its partial peptides, or salts thereof (hereinafter sometimes merely referred to as the protein of the present invention) may be manufactured by publicly known methods for manufacturing antibodies or antisera, using as antigens the protein of the present invention.

[Preparation of Monoclonal Antibody]

(a) Preparation of Monoclonal Antibody-producing Cells

The protein of the present invention is administered to a mammal either solely or together with carriers or diluents to the site where the production of antibody is possible by the administration. In order to potentiate the antibody productivity upon the administration, complete Freund's adjuvants or incomplete Freund's adjuvants may be administered. The administration is usually carried out once in every two to six weeks approximately 2 to 10 times in total. Examples of the applicable mammal are monkeys, rabbits, dogs, guinea pigs, mice, rats, sheep and goats, With mice and rats being preferred.

In the preparation of monoclonal antibody-producing cells, warm-blooded animals, e.g., mice, immunized with an antigen wherein the antibody titer is noted is s selected, then the spleen or lymph node is collected after 2 to 5 days from the final immunization and antibody-producing cells contained therein are fused with myeloma cells to give monoclonal antibody-producing hybridomas. Measurement of the antibody titer in antisera may be made, for example, by reacting a labeled form of the protein, which will be described later, with the antiserum followed by assaying the binding activity of the labeling agent bound to the antibody. The fusion may be operated, for example, by the known Koehler and Milstein method [Nature, 256, 495, (1975)]. Examples of the fusion accelerator are polyethylene glycol (PEG), Sendai virus, etc., of which PEG is preferably employed.

Examples of the myeloma cells are NS-1, P3U1, SP2/0, etc. In particular, P3U1 is preferably employed. A preferred ratio of the count of the antibody-producing cells used (spleen cells) to the count of myeloma cells is within a range of approximately 1:1 to 20:1. When PEG (preferably, PEG 1000 to PEG 6000) is added in a concentration of approximately 10 to 80% followed by incubating at about 20 to about 40° C., preferably at about 30 to about 37° C. for about 1 to about 10 minutes, an efficient cell fusion can be carried out.

Various methods can be used for screening of a monoclonal antibody-producing hybridoma. Examples of such methods include a method which comprises adding the supernatant of hybridoma to a solid phase (e.g., microplate) adsorbed with the protein etc. as an antigen directly or together with a carrier, adding an anti-immunoglobulin antibody (when mouse cells are used for the cell fusion, an anti-mouse immunoglobulin antibody is used) labeled with a radioactive substance or an enzyme, or Protein A and detecting the monoclonal antibody bound to the solid phase, and a method which comprises adding the supernatant of hybridoma to a solid phase adsorbed with an anti-immunoglobulin antibody or Protein A, adding the protein labeled with a radioactive substance or an enzyme and detecting the monoclonal antibody bound to the solid phase.

The monoclonal antibody-producing hybridoma can be selected by publicly known methods or by modifications of these methods. In general, the selection can be effected in a medium for animal cells supplemented with HAT (hypoxanthine, aminopterin and thymidine). Any selection and growth medium can be employed as far as the hybridoma can grow therein. For example, RPMI 1640 medium containing 1% to 20%, preferably 10% to 20% fetal cow serum, GIT medium (Wako Pure Chemical Industries, Ltd.) containing 1% to 10% fetal cow serum, a serum free medium for cultivation of a hybridoma (SFM-101, Nissui Seiyaku Co., Ltd.) and the like can be used for the selection and growth medium. The cultivation is carried out generally at 20° C. to 40° C., preferably at about 37° C., for 5 days to 3 weeks, preferably 1 to 2 weeks. The cultivation can be conducted normally in 5% CO₂. The antibody titer of the culture supernatant of hybridomas can be determined as in the assay for the antibody titer in antisera described above.

(b) Purification of Monoclonal Antibody

Separation and purification of the monoclonal antibody can be carried out by methods applied to conventional separation and purification of immunoglobulins, as in the conventional methods for separation and purification of polyclonal antibodies [e.g., salting-out, alcohol precipitation, isoelectric point precipitation, electrophoresis, adsorption and desorption with ion exchangers (e.g., DEAE), ultracentrifugation, gel filtration, or a specific purification method which comprises collecting only an antibody with an activated adsorbent such as an antigen-binding solid phase, Protein A, Protein G, etc. and dissociating the binding to obtain the antibody].

[Preparation of Polyclonal Antibody]

The polyclonal antibody of the present invention can be manufactured by publicly known methods or modifications thereof. For example, a complex of immunogen (antigen consisting of the protein of the present invention) and a carrier protein is prepared, and a mammal is immunized with the complex in a manner similar to the method described above for the manufacture of monoclonal antibodies. The product containing the antibody to the protein of the present invention is collected from the immunized animal followed by separation and purification of the antibody.

In the complex of an immunogen and a carrier protein used to immunize a mammal, the type of carrier protein and the mixing ratio of a carrier to hapten may be any type and in any ratio, as long as the antibody is efficiently produced to the hapten immunized by crosslinking to the carrier. For example, bovine serum albumin, bovine thyroglobulins, keyhole limpet hemocyanin, etc. is coupled to hapten in a carrier-to-hapten weight ratio of approximately 0.1 to 20, preferably about 1 to about 5.

A variety of condensing agents can be used for the coupling of a carrier to hapten. Glutaraldehyde, carbodiimide, maleimide activated ester, activated ester reagents containing thiol group or dithiopyridyl group, etc. are used for the coupling.

The condensation product is administered to warm-blooded animals either solely or together with carriers or diluents to the site in which the antibody can be produce by the administration. In order to potentiate the antibody productivity upon the administration, complete Freund's adjuvant or incomplete Freund's adjuvant may be administered. The administration is usually made once approximately in every 2 to 6 weeks and about 3 to about 10 times in total.

The polyclonal antibody can be collected from the blood, ascites, etc., preferably from the blood of mammals immunized by the method described above.

The polyclonal antibody titer in antiserum can be assayed by the same procedure as that for the determination of serum antibody titer described above. The separation and purification of the polyclonal antibody can be carried out, following the method for the separation and purification of immunoglobulins performed as applied to the separation and purification of monoclonal antibodies described hereinabove.

Hereinafter, the DNA that controls the production of Aβ (DNA of the present invention), the antisense DNA having the base sequence complementary to the DNA, the transformant by a recombinant vector containing the DNA, the protein encoded by the DNA, its partial peptides or salts thereof (protein of the present invention) and the antibodies of the present invention are explained below, in terms of use thereof.

(1) Tissue Marker of Alzheimer's Disease

The protein of the present invention is expressed also in the brain of patients with Alzheimer's disease, and thus can be used as a tissue marker for Abzeimer's disease. Also, the protein can be used to detect or fractionate a peptide, protein or DNA that selectively binds to the protein of the present invention.

(2) Therapeutic/Preventive Agents for Various Diseases with which the Protein of the Invention is Associated

The protein of the present invention having the activity of increasing the production of Aβ and its dominant negative variants can be used as drugs for the treatment/prevention of diseases, in which the activity or expression level is activated in patients with, for example, (1) neurodegenerative disorders (e.g., senile dementia, Alzheimer's disease, Down's syndrome, Parkinson's disease, Creutzfeldt-Jacob disease, amyotrophic sclerosis on lateral fasciculus of spinal, diabetic neuropathy, etc.), (2) neurological disorders caused by cerebrovascular disorders (e.g., cerebral infarction, encephalorrhagia, cerebrovascular disorders accompanied by cerebral arteriosclerosis, etc.), a head injury or an injury of spinal cord, sequelae of encephalitis or cerebral palsy, (3) memory impairment (e.g., senile dementia, amnesia, etc.), (4) mild cognitive impairment (M.C.I.), or (5) psychiatric disorders (e.g., depression, panic disorder, schizophrenia, etc.) or the like, preferably in patients with Alzheimer's disease.

The dominant negative variants described above may be any of variants, in which a part of the amino acid sequence is substituted or deleted, or amino acids are added to the amino acid sequence, or with a combination of substitution, deletion and addition, and in which the activity of increasing the production of Aβ is lost or attenuated.

The dominant negative variants may be prepared by means of conventional genetic engineering. For example, the dominant negative variants may be prepared by converting the base sequence of the DNA encoding the protein of the present invention in accordance with publicly known methods such as the Gapped duplex method or the Kunkel method, or modifications thereof, using publicly known kits available as, e.g., Mutan™-G or Mutan™-K (both manufactured by Takara Shuzo Co., Ltd.), etc., and following the process of manufacturing the protein of the present invention described above.

By assaying the Aβ production-increasing activity of the dominant negative variant or the DNA encoding the same by the screening method of the present invention, it is confirmed that the activity of increasing the production of Aβ is lost or attenuated.

(3) Method of Screening Drug Candidate Compounds for Various Diseases

The compound or its salt that inhibits (suppresses) the function (activity) of the protein of the present invention having the activity of increasing the production of Aβ can be used as pharmaceuticals for the treatment/prevention, etc. for, e.g., (1) neurodegenerative disorders (e.g., senile dementia, Alzheimer's disease, Down's syndrome, Parkinson's disease, Creutzfeldt-Jacob disease, amyotrophic sclerosis on lateral fasciculus of spinal, diabetic neuropathy, etc.), (2) neurological disorders caused by cerebrovascular disorders (e.g., cerebral infarction, encephalorrhagia, cerebrovascular disorders accompanied by cerebral arteriosclerosis, etc.), a head injury or an injury of spinal cord, sequelae of encephalitis or cerebral palsy, (3) memory impairment (e.g., senile dementia, amnesia, etc.), (4) mild cognitive impairment (M.C.I.), or (5) psychiatric disorders (e.g., depression, panic disorder, schizophrenia, etc.) or the like, preferably Alzheimer's disease.

Thus the protein and its DNA of the present invention are useful as a reagent for screening a compound that inhibits the function (activity) of the protein of the present invention.

That is, the present invention provides the method of screening the compound that inhibits (suppresses) the function (activity) of the protein of the present invention (hereinafter sometimes merely referred to as the inhibitor), which comprises using the protein of the present invention, its DNA, the antisense DNA complementarily binding to the DNA, the transformant transformed by a recombinant vector containing the DNA, or the antibody of the present invention.

Furthermore, the screening kit of the present invention comprises the protein of the present invention, its DNA, the antisense DNA complementarily binding to the DNA, the transformant transformed by a recombinant vector containing the DNA, or the antibody of the present invention.

Specifically, the screening method described above includes the following.

(3-1) Method of Screening a Compound that Inhibits the Reaction of the Protein of the Invention with Tissues, Cells or Membrane Fractions Thereof

(i) It is a method of screening the compound or its salt that inhibits the reactivity of the protein of the invention with tissues, cells or membrane fractions thereof, which comprises comparing (i) the case where the protein of the present invention is brought in contact with tissues, cells or membrane fractions thereof, with which the protein of the present invention is reactive, and (ii) the case where the protein of the present invention and a test compound are brought in contact with tissues, cells or membrane fractions thereof, with which the protein of the present invention is reactive.

As the cells described above, the established cells or primary culture system may be used.

For the cells or tissues, there may be used any cells derived from human and other warm-blooded animals (e.g., guinea pig, rat, mouse, fowl, rabbit, swine, sheep, bovine, monkey, etc.), (e.g., nerve cells, endocrine cells, neuroendocrine cells, glial cells, β cells of pancreas, bone marrow cells, hepatocytes, splenocytes, mesangial cells, epidermic cells, epithelial cells, endothelial cells, fibroblasts, fibrocytes, myocytes, fat cells, immune cells (e.g., macrophage, T cells, B cells, natural killer cells, mast cells, neutrophils, basophils, eosinophils, monocytes, dendritic cells), megakaryocytes, synovial cells, chondrocytes, bone cells, osteoblasts, osteoclasts, mammary gland cells, or interstitial cells; or the corresponding precursor cells, stem cells, cancer cells, etc.); or any tissues where such cells are present, such as brain or any of brain regions (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gall-bladder, bone marrow, adrenal gland, skin, muscle, lung, gastrointestinal tract (e.g., large intestine and small intestine), blood vessel, heart, thymus, spleen, submandibular gland, peripheral blood, prostate, testis (testicle), ovary, placenta, uterus, bone, joint, skeletal muscle, etc.

In the screening method of the present invention, the aforesaid reactivity is assayed on a binding amount, a cell stimulating activity, a tissue stimulating activity, etc., and comparison is made. To assay the binding amount, a measuring instrument such as Biacore, etc. or a marker ligand may be used occasionally. In the latter case, the protein of the present invention labeled with, e.g., [³H], [¹²⁵I], [¹⁴C], [³⁵S], a fluorescent dye, a fluorescent protein, biotin, an enzyme such as β-galactosidase or peroxidase, etc., a peptide called a tag such as FLAG, etc., is available.

Where the binding amount of the protein of the present invention containing the amino acid sequence represented by SEQ ID NO: 1, SEQ ID NO: 2 or SEQ ID NO: 3 is assayed, tissues or cells, or membrane fractions or crude purification fractions thereof, etc. containing syndecan-4, presenilin, or a protein called FLAP, or analog proteins thereof, respectively, may also be used. Any cell stimulating activity may be used, so long as the activity is accompanied by a biochemical change of the cells. Examples of such an activity include arachidonic acid metabolites, acetylcholine release, intracellular Ca²⁺ release, intracellular cAMP production, intracellular cGMP production, inositol phosphate production, pH reduction in extracellular fluids, changes in cell membrane potential, K⁺ channel function, phosphorylation of intracellular proteins, activation of c-fos, activation of NFκB, Ca level in the endoplasmic reticulum, capacitating calcium entry (CCE), endoplasmic reticulum stress, induction of a molecular chaperone accompanied by endoplasmic reticulum stress, tau phosphorylation, axonal transport, kinesin-dependent APP axonal transport, NO production, apoptosis, a cell growth activity, a cell adhesion activity, a cell migration activity, production of a physiologically active substance specifically produced by the cells, etc. Any tissue stimulating activity may be used, as long as it is accompanied by a biochemical or physiological change in the tissues, and the activity includes, e.g., a contraction or relaxation activity.

(3-2) Method of Screening a Promoter for or Inhibitor Against the Enzyme Activity of the Protein of the Present Invention

Where the protein of the present invention has an enzyme activity or is somehow associated an enzyme activity, the compound that promotes the enzyme activity of the protein of the present invention (hereinafter merely referred to as the promoter) or the compound that inhibits the activity (hereinafter merely referred to as the inhibitor) can be screened, using the activity as an indicator. For example, the protein of the present invention containing the amino acid sequence represented by SEQ ID NO: 3 corresponds to the N-terminal partial sequence of 5-lipoxygenase, and thus can be used to screen the promoter for or inhibitor against the 5-lipoxygenase activity.

(3-3) Method of Screening a Promoter for or Inhibitor Against the Activity of the Protein of the Present Invention Using a Transformant

Using a transformant by a recombinant vector containing the DNA encoding the protein of the present invention or its partial peptide, the compound that promotes the enzyme activity of the protein of the present invention (hereinafter merely referred to as the promoter) or the compound that inhibits the activity (hereinafter merely referred to as the inhibitor) can be screened, using as an indicator a biochemical change of the transformant attributed to the DNA introduced.

As the transformant, yeast and cells are preferably used. Where cells are used, either the established cells or primary culture system may be used as the parent strain used and furthermore, various cells described in (3-1) above may also be used. Any change is usable as the indicator of chemical change of the transformant attributed to the said DNA introduced, as long as the change is detectable.

For example, a substance (e.g., Aβ) specifically produced by the transformant may be utilized as the indicator.

To assay Aβ, various methods are used, and immunochemical methods using an Aβ-specific antibody are preferred. These methods used include immunoprecipitation, western blotting, enzyme immunoassay and sandwich enzyme immunoassay, or a combination thereof. Any parent strain is usable to prepare the transformant, as long as it can produce the amount of βAPP sufficient for detection even though it is the strain which that βAPP is expressed endogenously, βAPP or its fragment is exogenously introduced, the γ-secretase activity is endogenously possessed, or presenilin or the like is introduced to eventually exert the γ-secretase activity. The βAPP or presenilin introduced may contain FAD-derived mutations. Furthermore, where the cell line designed to activate the expression of a chemical resistant drug such as puromycin, etc. by promoting the production of Aβ from the βAPP fragment is used as the parent strain, the screening of compound can be performed using as an indicator the chemical resistance of puromycin, etc., in addition to the assay of Aβ.

As the substance specifically produced by the transformant attributed to the introduced DNA, secretory APP may also be used, in addition to Aβ. Moreover, particular attention may be drawn to Aβ42 in the Aβ family.

Furthermore, an interactive change with a substance functionally associated with the protein of the present invention, for example, a ligand, a substrate, a substance to be transported or a complex-forming factor may be assayed as an indicator of the aforesaid biochemical change of the transformant attributed to the introduced DNA.

The factor which forms a complex with the protein of the present invention containing the amino acid sequence represented by SEQ ID NO: 1, SEQ ID NO: 2, SEQ ID NO: 3 or SEQ ID NO: 22 includes syndecan4, presenilin, a protein called FLAP, or analog proteins thereof, respectively.

Furthermore, as the indicator of the aforesaid biochemical change of the transformant attributed to the introduced DNA described above, there may be assayed, for example, arachidonic acid metabolites, acetylcholine release, intracellular Ca²⁺ release, intracellular cAMP production, intracellular cGMP production, inositol phosphate production, pH reduction in extracellular fluids, changes in cell membrane potential, K⁺ channel function, phosphorylation of intracellular proteins, activation of c-fos, activation of NFκB, Ca level in the endoplasmic reticulum, capacitating calcium entry (CCE), endoplasmic reticulum stress, induction of a molecular chaperone accompanied by endoplasmic reticulum stress, tau phosphorylation, axonal transport, kinesin-dependent APP axonal transport, NO production, apoptosis, a cell growth activity, a cell adhesion activity, a cell migration activity, etc.

Furthermore, migration of the protein of the present invention or a certain specific protein into the nucleus, endoplasmic reticulum, Golgi body, mitochondria, endosome, lysosome, or membranes thereof, proteasome or cell membranes may be measured as the indicator of the said biochemical change of the transformant described above.

The specific protein includes syndecan-4, presenilin, a protein called FLAP, or analog proteins thereof, which is a factor to form a complex with the protein containing the amino acid sequence represented by SEQ ID NO: 1, SEQ ID NO: 2, SEQ ID NO: 3 or SEQ ID NO: 22, respectively. For its detection, a label is metabolically introduced using a radioactively labeled amino acid, etc. followed by immunoprecipitation using an antibody, or a chimeric protein to which a fluorescent protein such as GFP or a tag such as FLAG, etc. is bound is expressed in these proteins thereby to measure the protein migration using the fluorescence or tag as the indicator.

(3-4) Method of Screening a Compound that Inhibits Expression of the Protein of the Present Invention

The protein of the present invention, its DNA, the antisense DNA complementarily binding to the DNA, the transformant by a recombinant vector containing the DNA, or the antibody of the present invention can be used to screen the compound that inhibits expression of the protein of the present invention.

For materials to be used, the cells in which the protein of the present invention is expressed are employed, but tissues, animals, etc. may also be used. In this case, the established cells or primary culture system may also be used and various cells or tissues described in (3-1) above may further be used. The animals described above also include knockout animals transfected with a reporter gene, which will be later described. The expression level of the protein of the present invention can be assayed by publicly known methods such as immunochemical assays using an antibody, etc.; alternatively, mRNA of the protein of the present invention may also be assayed publicly known methods using the northern hybridization technique, RT-PCR or TaqMan PCR.

Moreover, the compound that promotes or inhibits the promoter activity can be screened by reporter gene assay, using the promoter region (a promoting promoter, a suppressing promoter, etc.) of the gene encoding the protein of the present invention in combination with a reporter gene. In this case, the established cells or primary culture system may also be used as the parent strain, and various cells or tissues described in (3-1) above may further be used. As the reporter gene, preferably used are chloramphenicol acetyltransferase (CAT), β-galactosidase, luciferase, growth factor, β-glcuronidase, alkaline phosphatase, Green fluorescent protein (GFP), β-lactamase, etc. Vector construction of these reporter genes and their assay methods may be carried out according to publicly known techniques (e.g., Molecular Biotechnology, 13, 29-43, 1999).

(4) Diagnostic Using the Antibody of the Present Invention

The antibody of the present invention is prepared and can be used for diagnosis of various diseases, for example, (1) neurodegenerative disorders (e.g., senile dementia, Alzheimer's disease, Down's syndrome, Parkinson's disease, Creutzfeldt-Jacob disease, amyotrophic sclerosis on lateral fasciculus of spinal, diabetic neuropathy, etc.), (2) neurological disorders caused by cerebrovascular disorders (e.g., cerebral infarction, encephalorrhagia, cerebrovascular disorders accompanied by cerebral arteriosclerosis, etc.), a head injury or an injury of spinal cord, sequelae of encephalitis or cerebral palsy, (3) memory impairment (e.g., senile dementia, amnesia, etc.), (4) mild cognitive impairment (M.C.I.), or (5) psychiatric disorders (e.g., depression, panic disorder, schizophrenia, etc.) or the like, preferably Alzheimer's disease. Also, the protein of the present invention can be quantified using antibodies thereto, and also detected by tissue staining, or the like. For this purpose, an antibody molecule itself may be used, or F(ab′)₂, Fab′ or Fab fractions of the antibody molecule may also be used. The antibodies may be monoclonal antibodies, polyclonal antibodies, human-mouse chimeric antibodies, human antibodies or human antibodies genetically engineered. Human antibodies may be prepared by cell fusion technique using human myeloma cells, or by immunizing human immunoglobulin gene-transfected mouse followed by cell fusion of immunocompetent cells of the mouse to myeloma cells (K. Tomizuka et. al., Proc. Nad. Acad. Sci. 97, 722-727, 2000). The human antibody genetically engineered also includes a single-chain antibody with crosslinked V_{H} and V_{L} regions.

The method for quantifying the protein of the present invention using the antibody of the present invention is not particularly limited. Any assay method can be used, so long as the amount of antibody, antigen, or antibody-antigen complex corresponding to the amount of antigen (e.g., the amount of the protein of the present invention) in a test fluid can be detected by chemical or physical means and the amount of the antigen can be calculated from a standard curve prepared from standard solutions containing known amounts of the antigen. For example, nephrometry, competitive methods, immunometric methods, and sandwich methods are appropriately used, with the sandwich methods described below being most preferable in terms of sensitivity and specificity. In applying these individual immunoassay methods to the method for quantification of the present invention, it is unnecessary to set forth particular conditions, procedures, etc. One skilled in the art may add his ordinary technical consideration to the conventional conditions and procedures in the respective methods to make up the assay system of the polypeptide of the present invention. For the details of these general technical means, reference can be made to the following reviews and texts.

For example, Hiroshi Irie, ed. “Radioimmunoassay” (Kodansha, published in 1974), Hiroshi Irie, ed. “Sequel to the Radioimmunoassay” (Kodansha, published in 1979), Eiji Ishikawa, et al. ed. “Enzyme immonoassay” (Igakushoin, published in 1978), Eiji Ishikawa, et al. ed. “Immunoenzyme assay” (2nd ed.) (Igakushoin, published in 1982), Eiji Ishikawa, et al. ed. “Immunoenzyme assay” (3rd ed.) (Igakushoin, published in 1987), Methods in ENZYMOLOGY, Vol. 70 (Immunochemical Techniques (Part A)), ibid., Vol. 73 (Immunochemical Techniques (Part B)), ibid., Vol. 74 (Immunochemical Techniques (Part C)), ibid., Vol. 84 (Immunochemical Techniques (Part D: Selected Immunoassays)), ibid., Vol. 92 (Immunochemical Techniques (Part E: Monoclonal Antibodies and General Immunoassay Methods)), ibid., Vol. 121 (Immunochemical Techniques (Part I: Hybridoma Technology and Monoclonal Antibodies)) (all published by Academic Press Publishing).

(5) Pharmaceutical Comprising the Antibody of the Present Invention

The antibody of the present invention, which has the action of neutralizing the activity of the protein of the present invention to increase the production of Aβ can be used as a pharmaceutical such as a therapeutic/preventive drug for, e.g., diseases caused by overexpression of the protein, for example, (1) neurodegenerative disorders (e.g., senile dementia, Alzheimer's disease, Down's syndrome, Parkinson's disease, Creutzfeldt-Jacob disease, amyotrophic sclerosis on lateral fasciculus of spinal, diabetic neuropathy, etc.), (2) neurological disorders caused by cerebrovascular disorders (e.g., cerebral infarction, encephalorrhagia, cerebrovascular disorders accompanied by cerebral arteriosclerosis, etc.), a head injury or an injury of spinal cord, sequelae of encephalitis or cerebral palsy, (3) memory impairment (e.g., senile dementia, amnesia, etc.), (4) mild cognitive impairment (M.C.I.), or (5) psychiatric disorders (e.g., depression, panic disorder, schizophrenia, etc.) or the like, preferably Alzheimer's disease.

The antibodies may be monoclonal antibodies, polyclonal antibodies, human-mouse chimeric antibodies, human antibodies or human antibodies genetically engineered. Human antibodies may be prepared by cell fusion technique using human myeloma cells, or by immunizing human immunoglobulin gene-transfected mouse followed by cell fusion of immunocompetent cells of the mouse to myeloma cells. The human antibody genetically engineered also includes a single-chain antibody with crosslinked V_{H} and V_{L} regions.

(6) Vaccine Using the Protein of the Present Invention

By using as a vaccine by itself or together with a carrier protein for immunization, the protein of the present invention can be used as a progression-preventive agent or therapeutic agent for various diseases, for example, (1) neurodegenerative disorders (e.g., senile dementia, Alzheimer's disease, Down's syndrome, Parkinson's disease, Creutzfeldt-Jacob disease, amyotrophic sclerosis on lateral fasciculus of spinal, diabetic neuropathy, etc.), (2) neurological disorders caused by cerebrovascular disorders (e.g., cerebral infarction, encephalorrhagia, cerebrovascular disorders accompanied by cerebral arteriosclerosis, etc.), a head injury or an injury of spinal cord, sequelae of encephalitis or cerebral palsy, (3) memory impairment (e.g., senile dementia, amnesia, etc.), (4) mild cognitive impairment (M.C.I.), or (5) psychiatric disorders (e.g., depression, panic disorder, schizophrenia, etc.) or the like, preferably Alzheimer's disease. As the carrier protein, any carrier protein is usable so long as it is highly safe to the living body; for example, tetanus toxin or the like is employed.

(7) Gene Diagnosis Associated with the Protein of the Present Invention

When abnormalities (genetic abnormalities) are found, the information on the properties of DNA (including the promoter region, exon and intron) encoding the protein of the present invention or mRNA means to detect abnormalities such as damages of the DNA or the mRNA , mutations, reduced expression, increase in the number of copies, overexpression, etc., associated with, e.g., (1) neurodegenerative disorders (e.g., senile dementia, Alzheimer's disease, Down's syndrome, Parkinson's disease, Creutzfeldt-Jacob disease, amyotrophic sclerosis on lateral fasciculus of spinal, diabetic neuropathy, etc.), (2) neurological disorders caused by cerebrovascular disorders (e.g., cerebral infarction, encephalorrhagia, cerebrovascular disorders accompanied by cerebral arteriosclerosis, etc.), a head injury or an injury of spinal cord, sequelae of encephalitis or cerebral palsy, (3) memory impairment (e.g., senile dementia, amnesia, etc.), (4) mild cognitive impairment (M.C.I.), or (5) psychiatric disorders (e.g., depression, panic. disorder, schizophrenia, etc.) or the like, preferably Alzheimer's disease, and thus useful for gene diagnosis. With regard to the mRNA, the increased or decreased expression of splice variants or the mutagenesis by mRNA editing (C. M. Niswender et. al., Ann. N.Y. Acad. Sci. 861, 38-48, 1998) is further considered. Also, the information on the chromosomal loci can be utilized for studies of hereditary diseases in which the DNA of the present participates. The gene diagnosis described above using the DNA encoding the protein of the present invention can be performed, for example, by publicly known northern hybridization or PCR-SSCP (Genomics, 5, 874-879 (1989), Proceedings of the National Academy of Sciences of the United States of America, 86, 2766-2770 (1989)), DNA microarray (Science, 270 467-470 (1995), or other methods (Jikken Igaku (Experimental Medicine, 18 (14),1894-1906, 2000), etc. Where the increased or decreased expression of the gene or mutation of the DNA is detected by any of the methods described above, it can be diagnosed that one tends to suffer from various diseases, e.g., (1) neurodegenerative disorders (e.g., senile dementia, Alzheimer's disease, Down's syndrome, Parkinson's disease, Creutzfeldt-Jacob disease, amyotrophic sclerosis on lateral fasciculus of spinal, diabetic neuropathy, etc.), (2) neurological disorders caused by cerebrovascular disorders (e.g., cerebral infarction, encephalorrhagia, cerebrovascular disorders accompanied by cerebral arteriosclerosis, etc.), a head injury or an injury of spinal cord, sequelae of encephalitis or cerebral palsy, (3) memory impairment (e.g., senile dementia, amnesia, etc.), (4) mild cognitive impairment (M.C.I.), or (5) psychiatric disorders (e.g., depression, panic disorder, schizophrenia, etc.) or the like, preferably Alzheimer's disease; or it is highly likely for one to suffer from these disease in the future, or the like.

Especially in recent years, polymorphic markers called SNPs (single nucleotide polymorphisms) made a new appearance as a critically important tool to investigate disease-associated genes, and have been drawing a sudden attention, as regulating the vulnerability to (difficulty to suffer from) diseases, or as also affecting the difference in response to drugs or the difference in side effects.

Typing of SNPs includes, depending on its specific purpose, the direct sequencing technique, the Invader method, the Sniper method, the MALDI-TOF/MS method, the oligo-SNP chip, etc. (Jikken Igaku (Experimental Medicine), 18 (12), 2000).

The SNPs present in the DNA (including the promoter region, exon and intron) encoding the protein of the present invention, which were found by these techniques, are analyzed solely or in combination with SNPs on the other genes or the DNA of the present invention, and thus important for determining vulnerability to, predicting the onset of, or diagnosis of, e.g., (1) neurodegenerative disorders (e.g., senile dementia, Alzheimer's disease, Down's syndrome, Parkinson's disease, Creutzfeldt-Jacob disease, amyotrophic sclerosis on lateral fasciculus of spinal, diabetic neuropathy, etc.), (2) neurological disorders caused by cerebrovascular disorders (e.g., cerebral infarction, encephalorrhagia, cerebrovascular disorders accompanied by cerebral arteriosclerosis, etc.), a head injury or an injury of spinal cord, sequelae of encephalitis or cerebral palsy, (3) memory impairment (e.g., senile dementia, amnesia, etc.), (4) mild cognitive impairment (M.C.I.), or (5) psychiatric disorders (e.g., depression, panic disorder, schizophrenia, etc.) or the like, preferably Alzheimer's disease.

(8) Pharmaceutical Comprising the Antisense DNA Associated with the Protein of the Present Invention

The antisense DNA containing the complementary base sequence to the DNA of the present invention that increases the production of Aβ, or a part of the DNA, capable of complementarily binding to the DNA and capable of suppressing expression of the DNA can suppress the in vivo functions of the DNA or the protein encoded by the DNA. Thus, the antisense DNA can be used as agents for the treatment/prevention of diseases, e.g., caused by overexpression of the protein to increase the production of Aβ, for example, (1) neurodegenerative disorders (e.g., senile dementia, Alzheimer's disease, Down's syndrome, Parkinson's disease, Creutzfeldt-Jacob disease, amyotrophic sclerosis on lateral fasciculus of spinal, diabetic neuropathy, etc.), (2) neurological disorders caused by cerebrovascular disorders (e.g., cerebral infarction, encephalorrhagia, cerebrovascular disorders accompanied by cerebral arteriosclerosis, etc.), a head injury or an injury of spinal cord, sequelae of encephalitis or cerebral palsy, (3) memory impairment (e.g., senile dementia, amnesia, etc.), (4) mild cognitive impairment (M.C.I.), or (5) psychiatric disorders (e.g., depression, panic disorder, schizophrenia, etc.) or the like, preferably Alzheimer's disease.

For example, the antisense DNA may be administered directly, or it is inserted into an appropriate vector such as a retrovirus vector, adenovirus vector, adenovirus-associated virus vector, etc. and then administered in a conventional manner. The antisense DNA may also be administered as it is; alternatively, the antisense DNA is prepared into pharmaceutical preparations together with pharmacologically acceptable carriers such as adjuvants to assist its uptake, which are then administered by gene gun or through a catheter such as a catheter with a hydrogel. Further alternatively, the antisense DNA is prepared into aerosol, which may also be intratracheally injected as an inhaler.

In addition, the antisense DNA may be used also as an oligonucleotide probe for diagnosis to examine the presence of the DNA of the present invention in tissues or cells or the state of its expression.

(9) Method for Assessment of the Preventive/Therapeutic Agent of Alzheimer's Disease Using DNA Transgenic Animal

The present invention provides a method for assessment of the preventive/therapeutic agent of Alzheimer's disease using a non-human mammal bearing an exogenous DNA encoding the protein of the present invention (hereinafter merely referred to as the exogenous DNA of the present invention) or its variant DNA (sometimes simply referred to as the exogenous variant DNA of the present invention).

Thus, the present invention provides:

(1) A non-human mammal bearing the exogenous DNA of the present invention or its variant DNA;

(2) The mammal according to (1), wherein the non-human mammal is a rodent;

(3) The mammal according to (2), wherein the rodent is mouse or rat; and,

(4) A recombinant vector bearing the exogenous DNA of the present invention or its variant DNA and capable of expressing in a mammal.

The non-human mammal bearing the exogenous DNA of the present invention or its variant DNA (hereinafter simply referred to as the DNA transgenic animal of the present invention) can be prepared by transfecting a desired DNA into an unfertilized egg, a fertilized egg, a spermatozoon, a germinal cell containing a primordial germinal cell thereof, or the like, preferably in the embryogenic stage in the development of a non-human mammal (more preferably in the single cell or fertilized cell stage and generally before the 8-cell phase), by standard means, such as the calcium phosphate method, the electric pulse method, the lipofection method, the agglutination method, the microinjection method, the particle gun method, the DEAE-dextran method etc. Also, it is possible to transfect the exogenous DNA of the present invention into a somatic cell, a living organ, a tissue cell, or the like by the DNA transfection methods, and utilize the transformant for cell culture, tissue culture, etc. In addition, these cells may be fused with the above-described germinal cell by a publicly known cell fusion method to create the DNA transgenic animal of the present invention.

Examples of the non-human mammal that can be used include bovine, swine, sheep, goats, rabbits, dogs, cats, guinea pigs, hamsters, mice, rats, and the like. Above all, preferred are rodents, especially mice (e.g., C₅₇BL/6 strain, DBA2 strain, etc. for a pure line and for a cross line, B6C3F₁ strain, BDF₁ strain B6D2F₁ strain, BALB/c strain, ICR strain, etc.) or rats (Wistar, SD, etc.), since they are relatively short in ontogeny and life cycle from a standpoint of creating model animals for human disease.

“Mammals” in a recombinant vector that can be expressed in the mammals include the aforesaid non-human mammals and human, etc.

The exogenous DNA of the present invention refers to the DNA of the present invention that is once isolated/extracted from mammals, not the DNA of the present invention inherently possessed by the non-human mammals.

The mutant DNA of the present invention includes mutants resulting from variation (e.g., mutation, etc.) in the base sequence of the original DNA of the present invention, specifically DNAs resulting from base addition, deletion, substitution with other bases, etc. and further including abnormal DNA.

The abnormal DNA is intended to mean such a DNA that expresses the abnormal protein of the present invention and exemplified by the DNA that expresses a protein to suppress the functions of the normal protein of the present invention.

The exogenous DNA of the present invention may be any one of those derived from a mammal of the same species as, or a different species from, the mammal as the target animal. In transfecting the DNA of the present invention into the target animal, it is generally advantageous to use the DNA as a DNA construct in which the DNA is ligated downstream a promoter capable of expressing the DNA in the target animal. For example, in the case of transfecting the human DNA of the present invention, a DNA-introduced mammal that expresses the DNA of the present invention to a high level, can be prepared by microinjecting a DNA construct (e.g., vector, etc.) ligated with the human DNA of the present invention into a fertilized egg of the target non-human mammal downstream various promoters, which are capable of expressing the DNA derived from various mammals (e.g., rabbits, dogs, cats, guinea pigs, hamsters, rats, mice, etc.) bearing the DNA of the present invention highly homologous to the human DNA.

As expression vectors for the protein of the present invention, there are Escherichia coli-derived plasmids, Bacillus subtilis-derived plasmids, yeast-derived plasmids, bacteriophages such as λ phage, retroviruses such as Moloney leukemia virus, etc., and animal viruses such as vaccinia virus, baculovirus, etc. Of these vectors, Escherichia coli-derived plasmids, Bacillus subtilis-derived plasmids, or yeast-derived plasmids, etc. are preferably used.

Examples of these promoters for regulating the DNA expression include (1) promoters for the DNA derived from viruses (e.g., simian virus, cytomegalovirus, Moloney leukemia virus, JC virus, breast cancer virus, poliovirus, etc.), and (2) promoters derived from various mammals (human, rabbits, dogs, cats, guinea pigs, hamsters, rats, mice, etc.), for example, promoters of albumin, insulin II, uroplakin II, elastase, erythropoietin, endothelin, muscular creatine kinase, glial fibrillary acidic protein, glutathione S-transferase, platelet-derived growth factor β, keratins K1, K10 and K14, collagen types I and II, cyclic AMP-dependent protein kinase βI subunit, dystrophin, tartarate-resistant alkaline phosphatase, atrial natriuretic factor, endothelial receptor tyrosine kinase (generally abbreviated as Tie2), sodium-potassium adenosine triphosphorylase (Na,K-ATPase), neurofilament light chain, metallothioneins I and IIA, metalloproteinase I tissue inhibitor, MHC class I antigen (H-2L), H-ras, renin, dopamine β-hydroxylase, thyroid peroxidase (TPO), protein chain elongation factor 1α (EF-1α), β actin, α and β myosin heavy chains, myosin light chains 1 and 2, myelin base protein, thyroglobulins, Thy-1, immunoglobulins, H-chain variable region (VNP), serum amyloid component P, myoglobin, troponin C, smooth muscle α actin, preproencephalin A, vasopressin, etc. Among others them, cytomegalovirus promoters, human protein elongation factor 1α (EF-1α) promoters, human and chicken β actin promoters etc., which protein can highly express in the whole body are preferred.

It is preferred that the vectors described above have a sequence for terminating the transcription of the desired mRNA in the DNA-introduced animal (generally called a terminator); for example, a sequence of each DNA derived from viruses and various mammals. SV40 terminator of the simian virus, and the like, are preferably used.

In addition, for the purpose of increasing the expression of the desired exogenous DNA to a higher level, the splicing signal and enhancer region of each DNA, a portion of the intron of an eukaryotic DNA may also be ligated at the 5′ upstream of the promoter region, or between the promoter region and the translational region, or at the 3′ downstream of the translational region, depending upon purposes.

The said translational region can be prepared by a conventional DNA engineering technique, in which the DNA is ligated downstream the aforesaid promoter and if desired, upstream the translation termination site, as a DNA construct capable of being expressed in the transgenic animal.

The exogenous DNA of the present invention is transfected at the fertilized egg cell stage in a manner such that the DNA is certainly present in all the germinal cells and somatic cells of the target mammal. The fact that the exogenous DNA of the present invention is present in the germinal cells of the animal prepared by DNA transfection means that all offspring of the prepared animal will maintain the exogenous DNA of the present invention in all of the germinal cells and somatic cells thereof. The offspring of the animal that inherits the exogenous DNA of the present invention also have the exogenous DNA in all of the germinal cells and somatic cells thereof.

The non-human mammal in which the normal exogenous DNA of the present invention has been transfected can be passaged as the DNA-bearing animal under ordinary rearing environment, by confirming that the exogenous DNA is stably retained by mating.

By the transfection of the exogenous DNA of the present invention at the fertilized egg cell stage, the DNA is retained to be excess in all of the germinal and somatic cells. The fact that the exogenous DNA of the present invention is excessively present in the germinal cells of the prepared animal after transfection means that the exogenous DNA of the present invention is excessively present in all of the germinal cells and somatic cells thereof. The offspring of the animal that inherits the exogenous DNA of the present invention have excessively the exogenous DNA of the present invention in all of the germinal cells and somatic cells thereof.

By obtaining a homozygotic animal having the transfected DNA in both of homologous chromosomes and mating a male and female of the animal, all offspring can be passaged to retain the DNA in an excess level.

In a non-human mammal bearing the normal DNA of the present invention wherein the normal DNA of the present invention has expressed to a high level, the non-human mammal promotes the function of endogenous normal DNA and sometimes may eventually develop the hyperfunction of the protein of the present invention or develop diseases with which the protein of the present invention associates, e.g., Alzheimer's disease or Alzheimer's disease-like pathological conditions (e.g., cerebral deposition of Aβ, PHF-tau, neuronal cell death, etc.). Therefore, the animal can be utilized as a pathologic model animal for such diseases. For example, using the normal DNA transgenic animal of the present invention, its tissues and its cells, it is possible to elucidate the mechanism of the hyperfunction of the protein of the present invention and the pathological mechanism of the disease associated with the protein of the present invention, e.g., Alzheimer's disease or Alzheimer's disease-like pathological conditions (e.g., cerebral deposition of Aβ, PHF-tau, neuronal cell death, etc.) and to determine how to treat the disease. It is also possible to make use of a screening test for agents for the treatment/prevention of Alzheimer's disease.

On the other hand, non-human mammal having the exogenous abnormal DNA of the present invention can be passaged under normal breeding conditions as the DNA-bearing animal by confirming the stable retaining of the exogenous DNA via crossing. In addition, the exogenous DNA to be subjected can be utilized as a starting material by inserting the desired exogenous DNA into the plasmid described above. The DNA construct with a promoter can be prepared by conventional DNA engineering techniques. The introduction of the abnormal DNA of the present invention at the fertilized egg cell stage is preserved to be present in all of the germinal and somatic cells of the mammals to be subjected. The fact that the abnormal DNA of the present invention is present in the germinal cells of the animal after DNA transfection means that all of the offspring of the prepared animal have the abnormal DNA of the present invention in all of the germinal and somatic cells. Such an offspring passaged the exogenous DNA of the present invention contains the abnormal DNA of the present invention in all of the germinal and somatic cells. A homozygous animal having the introduced DNA on both of homologous chromosomes can be acquired and then by mating these male and female animals, all the offspring can be bled to have the DNA.

Since non-human mammal having the abnormal DNA of the present invention may express the abnormal DNA of the present invention at a high level, the animal may be the function inactivation type inadaptability of the protein of the present invention by inhibiting the function of the endogenous normal DNA and can be utilized as its disease model animal. For example, using the abnormal DNA-introduced animal of the present invention, its tissues and its cells, it is possible to elucidate the mechanism of inadaptability of the protein of the present invention and to perform to study a method for treatment of this disease, as well as to utilize for a screening test.

More specifically, the transgenic animal of the present invention expressing the abnormal DNA of the present invention to a high level is also expected to serve as an experimental model for the elucidation of the mechanism of the functional inhibition (dominant negative effect) of normal protein by the abnormal protein of the present invention in the function inactive type inadaptability of the protein of the present invention.

The protein of the present invention is closely related to the production of Aβ, and its increased expression results in increasing the production of Aβ. On the other hand, this fact suggest the possibility that the protein in an expression level within the normal range could be associated with the physiological role of the γ-secretase complex containing βAPP or presenilin. Thus, the abnormal DNA-transgenic animal with the protein of the present invention and its tissues or cells are useful for investigations of agents for the treatment/prevention of Alzheimer's disease.

Other potential applications of two kinds of the transgenic animals described above include:

(1) use as a cell source for tissue culture;

(2) elucidation of the relation to the protein of the present invention that is specifically expressed or activated by the protein of the present invention, by direct analysis of DNA or RNA in tissues of the DNA-introduced animal of the present invention or by analysis of the protein tissues expressed by the DNA;

(3) research in the function of cells derived from tissues that are cultured usually only with difficulty, using cells of tissue bearing the DNA cultured by a standard tissue culture technique;

(4) screening of a drug that enhances the functions of cells using the cells described in (3) above; and,

(5) isolation and purification of the variant protein of the present invention and preparation of an antibody thereto.

Furthermore, clinical conditions of a disease associated wit the protein of the present invention, including the function inactive type inadaptability of the protein of the present invention can be determined using the DNA-introduced animal of the present invention. Also, pathological findings on each organ in a disease model associated with the protein of the present invention can be obtained in more detail, leading to the development of a new method for treatment as well as the research and therapy of any secondary diseases associated with the disease.

It is also possible to obtain a free DNA-transfected cell by withdrawing each organ from the DNA transgenic animal of the present invention, mincing the organ and degrading with a proteinase such as trypsin, etc., followed by establishing the line of culturing or cultured cells. Furthermore, the DNA-introduced animal of the present invention can serve as identification of cells capable of producing the protein of the present invention, and as studies on association with apoptosis, differentiation or propagation or on the mechanism of signal transduction in these properties to inspect any abnormality therein. Thus the DNA transgenic animal of the present invention can provide an effective research material for the protein of the present invention and for elucidating the function and effect thereof.

To develop a therapeutic drug for the treatment of diseases associated with the protein of the present invention, including the function inactive type inadaptability of the protein of the present invention, using the DNA-introduced animal of the present invention, an effective and rapid method for screening can be provided by applying the method for inspection and the method for quantification, etc. described above. It is also possible to investigate and develop a method for DNA therapy for the treatment of diseases associated with the protein of the present invention, using the DNA-introduced animal of the present invention or a vector capable of expressing the exogenous DNA of the present invention.

(10) Method for Assessment of the Preventive/Therapeutic Drug of Alzheimer's Disease Using Knockout Animal

The present invention provides a method for assessment of the preventive/therapeutic drug of Alzheimer's disease using a non-human mammal embryonic stem cell wherein the DNA encoding the protein of the present invention is inactivated and a non-human mammal deficient in expressing the DNA encoding the protein of the present invention. Hereinafter, the DNA encoding the protein of the present invention is sometimes merely referred to as the DNA of the present invention.

Thus, the present invention provides:

(1) A non-human embryonic stem cell in which the DNA of the present invention is inactivated;

(2) The embryonic stem cell according to (1), wherein the DNA is inactivated by introducing a reporter gene (e.g., β-galactosidase gene derived from Escherichia coli);

(3) The embryonic stem-cell according to (1), which is resistant to neomycin;

(4) The embryonic stem cell according to (1), wherein the non-human mammal is a rodent;

(5) The embryonic stem cell according to (4), wherein the rodent is mouse;

(6) A non-human mammal deficient in expressing the DNA of the present invention, wherein the DNA is inactivated;

(7) The non-human mammal according to (6), wherein the DNA is inactivated by inserting a reporter gene (e.g., β-galactosidase derived from Escherichia coli) therein and the reporter gene is capable of being expressed under control of a promoter for the DNA of the present invention;

(8) The non-human mammal according to (6), which is a rodent;

(9) The non-human mammal according to (8), wherein the rodent is mouse; and,

(10) A method for screening a compound or its salt that promotes or inhibits the promoter activity for the DNA of the present invention, which comprises administering a test compound to the mammal of (7) and detecting expression of the reporter gene.

The non-human mammal embryonic stem cell in which the DNA of the present invention is inactivated refers to a non-human mammal embryonic stem cell that suppresses the ability of the non-human mammal to express the DNA by artificially mutating the DNA of the present invention, or the DNA has no substantial ability to express the protein of the present invention (hereinafter sometimes referred to as the knockout DNA of the present invention) by substantially inactivating the activity of the protein of the present invention encoded by the DNA (hereinafter merely referred to as ES cell).

As the non-human mammal, the same examples as described above apply.

Techniques for artificially mutating the DNA of the present invention include deletion of a part or all of the DNA sequence and insertion of or substitution with other DNA, by genetic engineering. By these variations, the knockout DNA of the present invention may be prepared, for example, by shifting the reading frame of a codon or by disrupting the function of a promoter or exon.

Specifically, the non-human mammal embryonic stem cell in which the DNA of the present invention is inactivated (hereinafter merely referred to as the ES cell with the DNA of the present invention inactivated or the knockout ES cell of the present invention) can be obtained by, for example, isolating the DNA of the present invention that the desired non-human mammal possesses, inserting a DNA fragment having a DNA sequence constructed by inserting a drug resistant gene such as a neomycin resistant gene or a hygromycin resistant gene, or a reporter gene such as lacZ (β-galactosidase gene) or cat (chloramphenicol acetyltransferase gene), etc. into its exon site thereby to disable the functions of exon, or integrating to a chromosome of the subject animal by, e.g., homologous recombination, a DNA sequence which terminates gene transcription (e.g., polyA additional signal, etc.) in the intron between exons, thus inhibiting the synthesis of complete mRNA to eventually destroy the gene (hereinafter simply referred to as the targeting vector). The thus obtained ES cells are subjected to Southern hybridization analysis using a DNA sequence on or near the DNA of the present invention as a probe, or to PCR analysis using a DNA sequence on the targeting vector and another DNA sequence near the DNA of the present invention, which is not included in the targeting vector as primers, thereby to select the knockout ES cell of the present invention.

The parent ES cells to inactivate the DNA of the present invention by homologous recombination, etc. may be of a strain already established as described above, or may be originally established in accordance with a modification of the known method by Evans and Kaufman supra. For example, in the case of mouse ES cells, currently it is common practice to use ES cells of the 129 strain. However, since their immunological background is obscure, the C₅₇BL/6 mouse or the BDF₁ mouse (F₁ hybrid between C₅₇BL/6 and DBA/2), wherein the low ovum availability per C₅₇BL/6 in the C₅₇BL/6 mouse has been improved by crossing with DBA/2, may be preferably used, instead of obtaining a pure line of ES cells with the clear immunological genetic background and for other purposes. The BDF₁ mouse is advantageous in that, when a pathologic model mouse is generated using ES cells obtained therefrom, the genetic background can be changed to that of the C₅₇BL/6 mouse by back-crossing with the C₅₇BL/6 mouse, since its background is of the C₅₇BL/6 mouse, as well as being advantageous in that ovum availability per animal is high and ova are robust.

In establishing ES cells, blastocytes at 3.5 days after fertilization are commonly used. In the present invention, embryos are preferably collected at the 8-cell stage, after culturing until the blastocyte stage, the embryos are used to efficiently obtain a large number of early stage embryos.

Although the ES cells used may be of either sex, male ES cells are generally more convenient for generation of a germ cell line chimera and are therefore preferred. It is also desirable that sexes are identified as soon as possible to save painstaking culture time.

Methods for sex identification of the ES cell include the method in which a gene in the sex-determining region on the Y-chromosome is amplified by the PCR process and detected. When this method is used, one colony of ES cells (about 50 cells) is sufficient for sex-determination analysis, which karyotype analysis, for example G-banding method, requires about 10⁶ cells; therefore, the first selection of ES cells at the early stage of culture can be based on sex identification, and male cells can be selected early, which saves a significant amount of time at the early stage of culture.

Second selection can be achieved by, for example, number of chromosome confirmation by the G-banding method. It is usually desirable that the chromosome number of the obtained ES cells be 100% of the normal number. However, when it is difficult to obtain the cells having the normal number of chromosomes due to physical operation etc. in cell establishment, it is desirable that the ES cell be again cloned to a normal cell (e.g., in mouse cells having the number of chromosomes being 2n=40) after the gene of the ES cells is rendered knockout.

Although the embryonic stem cell line thus obtained shows a very high growth potential, it must be subcultured with great care, since it tends to lose its ontogenic capability. For example, the embryonic stem cell line is cultured at about 37° C. in a carbon dioxide incubator (preferably about 5% carbon dioxide and about 95% air, or about 5% oxygen, about 5% carbon dioxide and 90% air) in the presence of LIF (1-10000 U/ml) on appropriate feeder cells such as STO fibroblasts, treated with a trypsin/EDTA solution (normally about 0.001 to about 0.5% trypsin/about 0.1 to about 5 mM EDTA, preferably about 0.1% trypsin/1 mM EDTA) at the time of passage to obtain separate single cells, which are then seeded on freshly prepared feeder cells. This passage is normally conducted every 1 to 3 days; it is desirable that cells be observed at passage and cells found to be morphologically abnormal in culture, if any, be abandoned.

Where ES cells are allowed to reach a high density in mono-layers or to form cell aggregates in suspension under appropriate conditions, they will spontaneously differentiate to various cell types, for example, pariental and visceral muscles, cardiac muscle or the like [M. J. Evans and M. H. Kaufman, Nature, 292, 154, 1981; G. R. Martin, Proc. Natl. Acad. Sci. U.S.A., 78,7634, 1981; T. C. Doetschman et al, Journal of Embryology Experimental Morphology, 87, 27, 1985]. The cells deficient in expressing the DNA of the present invention, which are obtainable from the differentiated ES cells of the present invention, are useful for studying the protein of the present invention from an aspect of cell biology.

The non-human mammal deficient in expressing the DNA of the present invention can be identified from a normal animal by measuring the mRNA amount in the subject animal by a publicly known method, and indirectly comparing the levels of expression.

As the non-human mammal, the same examples supra apply.

With respect to the non-human mammal deficient in expressing the DNA of the present invention, the DNA of the present invention can be made knockout by transfecting a targeting vector, prepared as described above, to non-human mammal embryonic stem cells or oocytes thereof, and conducting homologous recombination in which a targeting vector DNA sequence, wherein the DNA of the present invention is inactivated by the transfection, is replaced with the DNA of the present invention on a chromosome of a non-human mammal embryonic stem cell or embryo thereof.

The cells with the DNA of the present invention knockout can be identified by the Southern hybridization analysis using a DNA sequence on or near the DNA of the present invention as a probe, or by PCR analysis using as primers a DNA sequence on the targeting vector and another DNA sequence, which is not included in the targeting vector. When non-human mammalian embryonic stem cells are used, a cell line wherein the DNA of the present invention is inactivated by homologous recombination is cloned; the resulting cloned cell line is injected to, e.g., a non-human mammalian embryo or blastocyst, at an appropriate stage such as the 8-cell stage. The resulting chimeric embryos are transplanted to the uterus of the pseudopregnant non-human mammal. The resulting animal is a chimeric animal composed of both cells having the normal locus of the DNA of the present invention and those having an artificially mutated locus of the DNA of the present invention.

When some germ cells of the chimeric animal have a mutated locus of the DNA of the present invention, an individual, which entire tissue is composed of cells having a mutated locus of the DNA of the present invention can be selected from a series of offspring obtained by crossing between such a chimeric animal and a normal animal, e.g., by coat color identification, etc. The individuals thus obtained are normally deficient in heterozygous expression of the protein of the present invention. The individuals deficient in homozygous expression of the protein of the present invention can be obtained from offspring of the intercross between the heterozygotes of the protein of the present invention.

When an oocyte is used, a DNA solution may be injected, e.g., to the prenucleus by microinjection thereby to obtain a transgenic non-human mammal having a targeting vector introduced in a chromosome thereof. From such transgenic non-human mammals, those having a mutation at the locus of the DNA of the present invention can be obtained by selection based on homologous recombination.

As described above, individuals in which the DNA of the present invention is rendered knockout permit passage rearing under ordinary rearing conditions, after the individuals obtained by their crossing have proven to have been knockout.

Furthermore, the genital system may be obtained and maintained by conventional methods. That is, by crossing male and female animals each having the inactivated DNA, homozygote animals having the inactivated DNA in both loci can be obtained. The homozygotes thus obtained may be reared so that one normal animal and two or more homozygotes are produced from a mother animal to efficiently obtain such homozygotes. By crossing male and female heterozygotes, homozygotes and heterozygotes having the inactivated DNA are proliferated and passaged.

The non-human mammal embryonic stem cell, in which the DNA of the present invention is inactivated, is very useful for preparing a non-human mammal deficient in expressing the DNA of the present invention.

Since the non-human mammal deficient in expressing the DNA of the present invention lacks various biological activities derived from the protein of the present invention, such an animal can be a disease model suspected of inactivated biological activities of the protein of the present invention and thus, offers an effective study to investigate causes for and therapy for these diseases.

The protein of the present invention is closely related to the production of Aβ, and its increased expression results in increasing the production of Aβ. On the other hand, this fact suggest the possibility that the protein of the present invention in an expression level within the normal range could be associated with the physiological role of the γ-secretase complex containing βAPP or presenilin. Thus, the non-human mammal deficient in expressing the DNA of the present invention and its tissues or cells is useful for investigations of agents for the treatment/prevention of Alzheimer's disease.

(10a) Method of Screening a Compound having Therapeutic/Preventive Effects on Diseases Caused by Deficiency, Damages, etc. of the DNA of the Present Invention

The non-human mammal deficient in expressing the DNA of the present invention can be employed for screening of a compound having therapeutic/prophylactic effects on diseases caused by deficiency, damages, etc. of the DNA of the present invention.

That is, the present invention provides a method of screening a compound having therapeutic/prophylactic effects for diseases caused by deficiency, damages, etc. of the DNA of the present invention, which comprises administering a test compound to the non-human mammal deficient in expressing the DNA of the present invention and observing and measuring a change occurred in the animal.

As the non-human mammal deficient in expressing the DNA of the present invention which can be employed for the screening method, the same examples as given hereinabove apply.

Examples of the test compounds include peptides, proteins, non-peptide compounds, synthetic compounds, fermentation products, cell extracts, vegetable extracts, animal tissue extracts, blood plasma, etc. These compounds may be novel compounds or publicly known compounds.

Specifically, the non-human mammal deficient in expressing the DNA of the present invention is treated with a test compound, comparison is made with an intact animal for control and a change in each organ, tissue, disease conditions, etc. of the animal is used as an indicator to assess the therapeutic/prophylactic effects of the test compound.

For treating an animal to be test with a test compound, for example, oral administration, intravenous injection, etc. are applied and the treatment is appropriately selected depending upon conditions of the test animal, properties of the test compound, etc. Furthermore, a dose of test compound to be administered can be appropriately chosen depending on method for administration, nature of test compound, etc.

In the screening method described above, when a test compound is administered to a test animal and found to improve Alzheimer's disease, the test compound can be selected to be a compound having a therapeutic/prophylactic effect for Alzheimer's disease.

(10b) Method of Screening a Compound that Promotes or Inhibits the Promoter Activity for the DNA of the Present Invention

The present invention provides a method of screening a compound or its salt that promotes or inhibits the promoter activity for the DNA of the present invention, which comprises administering a test compound to a non-human mammal deficient in expressing the DNA of the present invention and detecting expression of the reporter gene.

In the screening method supra, the non-human mammal deficient in expressing the DNA of the present invention is selected from the aforesaid non-human mammal deficient in expressing the DNA of the present invention, as an animal in which the DNA of the present invention is inactivated by introducing a reporter gene and the reporter gene is expressed under control of a promoter to the DNA of the present invention.

The same examples of the test compound apply to specific compounds used for the screening. As the reporter gene, the same specific examples apply to this screening method. Preferably employed are β-galactosidase (lacZ), soluble alkaline phosphatase gene, luciferase gene and the like.

Since the reporter gene is present under control of a promoter to the DNA of the present invention in the non-human mammal deficient in expressing the DNA of the present invention wherein the DNA of the present invention is substituted with the reporter gene, the promoter activity can be detected by tracing expression of a substance encoded by the reporter gene.

When a part of the DNA region encoding the peptide of the present invention is substituted with, e.g., β-galactosidase gene (lacZ) derived from Escherichia coli, β-galactosidase is expressed in a tissue where the peptide of the present invention should originally be expressed, instead of the peptide of the present invention. Thus, the state of expression condition of the peptide of the present invention can be readily observed in vivo of an animal by staining with a reagent, e.g., 5-bromo-4-chloro-3-indolyl-β-galactopyranoside (X-gal) which is substrate for β-galactosidase. Specifically, a mouse deficient in the peptide of the present invention, or its tissue slice section is fixed with glutaraldehyde, etc. After washing with phosphate buffered saline (PBS), the system is reacted with a staining solution containing X-gal at room temperature or about 37° C. for approximately 30 minutes to an hour. After the β-galactosidase reaction is terminated by washing the tissue preparation with 1 mM EDTA/PBS solution, the color formed is observed. Alternatively, mRNA encoding lacZ may be detected in a conventional manner.

The compound or salts thereof obtained using the aforesaid screening method are compounds that are selected from the test compounds described above and the compounds that promote or inhibit the promoter activity to the DNA of the present invention.

The compound or its salt that inhibits the promoter activity to the DNA of the present invention can inhibit expression of the peptide of the present invention thereby to inhibit the function of the peptide. Thus, the compound is useful as a drug for the prevention/treatment of diseases, e.g., Alzheimer's disease, etc.

Also, the compound or its salt that promotes the promoter activity of a suppressing promoter to the DNA of the present invention can suppress expression of the peptide of the present invention thereby to suppress the function of the peptide, and is thus useful as a drug for the prevention/treatment of diseases, e.g., Alzheimer's disease, etc.

As described above, the non-human mammal deficient in expressing the DNA of the present invention is extremely useful for screening compounds or salts thereof that promote or inhibit the activity of promoter to the DNA of the present invention, and can thus greatly contribute to investigations of causes for various diseases caused by failure to express the DNA of the present invention or to development of preventive/therapeutic agents for these diseases.

Moreover, when a so-called taansgenic animal (gene-transfected animal) is prepared by using a DNA containing the promoter region of the peptide of the present invention, ligating genes encoding various proteins downstream the same and injecting the genes into animal oocyte, the peptide can be specifically synthesized by the animal so that it becomes possible to investigate the activity in vivo. Furthermore, when an appropriate reporter gene is ligated to the promoter region described above to establish a cell line so as to express the gene, such can be used as a survey system of low molecular weight compounds that specifically promotes or suppresses the ability of producing the peptide itself of the present invention in vivo.

The compound obtained by the screening method of the present invention or compound derived therefrom (hereinafter sometimes merely referred to as the compound obtained by the screening method of the present invention) may be in the form of salts. As the salts of the compounds, there may be used salts with physiologically acceptable acids (e.g., inorganic acids, organic acids, etc.) or bases (e.g., alkali metal salts, etc.), preferably in the form of physiologically acceptable acid addition salts. Examples of such salts are salts with inorganic acids (e.g., hydrochloric acid, phosphoric acid, hydrobromic acid, sulfuric acid, etc.), salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid, etc.) and the like.

The pharmaceutical comprising the compound obtained by the screening method of the present invention or compound derived therefrom may be administered directly as it is or as an appropriate pharmaceutical composition. The pharmaceutical composition used for the administration described above contains a pharmacologically acceptable carrier with the aforesaid compounds or salts thereof, a diluent or excipient. Such a composition is provided in the preparation suitable for oral or parenteral administration.

That is, examples of the composition for oral administration include solid or s5 liquid preparations, specifically, tablets (including dragees and film-coated tablets), pills, granules, powdery preparations, capsules (including soft capsules), syrup, emulsions, suspensions, etc. Such a composition is manufactured by publicly known methods and contains a vehicle, a diluent or excipient conventionally used in the field of pharmaceutical preparations. Examples of the vehicle or excipient for tablets are lactose, starch, sucrose, magnesium stearate, etc.

Examples of the composition for parenteral administration that can be used are injections (e.g., subcutaneous, intravenous, intramuscular, intraperitoneal injections, etc.), external preparations (e.g., pernasal agent, percutaneous agent, ointment, etc.), suppositories (e.g., rectal agent, vaginal suppository, etc.), sustained release agents (e.g., sustained release microcapsules, etc.), pellets, drip, etc.

Since the pharmaceutical composition thus obtained is safe and low toxic, it can be administered to, e.g., mammal (e.g., human, rat, mouse, guinea pig, rabbit, sheep, swine, bovine, horse, cat, dog, monkey, etc.).

A dose of the compound or its salt to be administered varies depending upon particular disease, subject to be administered, route of administration, etc., and in oral administration to an adult patient (as 60 kg body weight) for the treatment of Alzheimer's disease, the compound is administered generally in a dose of approximately 0.01 to 1000 mg, preferably approximately 0.1 to 1000 mg, more preferably approximately 1.0 to 200 mg per day and most preferably approximately 1.0 to 50 mg. In parenteral administration, when administered to an adult patient (as 60 kg body weight) for the treatment of Alzheimer's disease, it is advantageous to administer the compound intravenously in the form of an injectable preparation in a daily dose of approximately 0.01 to 30 mg, preferably approximately 0.1 to 20 mg, more preferably approximately 0.1 to 10 mg, though the single dosage varies depending upon particular subject, particular disease, etc. For other animals, the compound can be administered in the corresponding dose with converting it into that for the 60 kg body weight.

The therapeutic/preventive agents for the diseases described above comprising the dominant negative variant to the protein of the present invention or the antibody of the present invention can be administered to a mammal (e.g., human, rat, rabbit, sheep, swine, bovine, cat, dog, monkey, etc.) orally or parenterally directly as a liquid preparation, or as a pharmaceutical composition in an appropriate preparation form. The dose varies depending on subject to be administered, target disease, conditions, route for administration, etc.; the dominant negative variant of the present invention or the antibody is advantageously administered through intravenous injection, normally in a single dose of approximately 0.01 to 20 mg/kg body weight, preferably about 0.01 to about 10 mg/kg body weight, and more preferably about 0.1 to about 5 mg/kg body weight, approximately in 1 to 5 times, preferably approximately 1 to 3 times, per day. For other parenteral administration and oral administration, the corresponding dose may be administered. When the conditions are extremely serious, the dose may be increased depending on the conditions.

The pharmaceutical comprising the dominant negative variant to the protein of the present invention or the antibody of the present invention may be manufactured as in the pharmaceutical comprising the compound or its salt, obtained by the screening method described above.

A dose of the pharmaceutical comprising the DNA encoding the dominant negative variant of the present invention or the antisense DNA of the present invention varies depending on target disease, subject to be administered, route for administration, etc. For example, where the antisense DNA is locally administered as an inhaler through intratracheal injection, the antisense DNA is administered to an adult (60 kg body weight) generally in a daily dose of about 0.1 to 100 mg.

The pharmaceutical comprising the DNA encoding the dominant negative variant of the present invention or the antisense DNA of the present invention may be manufactured as in the pharmaceutical comprising the compound or its salt, obtained by the screening method described above.

In the specification and drawings, the codes of bases and amino acids are shown by abbreviations and in this case, they are denoted in accordance with the IUPAC-IUB Commission on Biochemical Nomenclature or by the common codes in the art, examples of which are shown below. For amino acids that may have the optical isomer, L form is presented unless otherwise indicated.

DNA: deoxyribonucleic acid

cDNA: complementary deoxyribonucleic acid

A: adenine

T: thyrnine

G: guanine

C: cytosine

RNA: ribonucleic acid

mRNA: messenger ribonucleic acid

dATP: deoxyadenosine triphosphate

dTTP: deoxythynidine triphosphate

dGTP: deoxyguanosine triphosphate

dCTP: deoxycytidine triphosphate

ATP: adenosine triphosphate

EDTA: ethylenediaminetetraacetic acid

SDS: sodium dodecyl sulfate

Gly: glycine

Ala: alanine

Val: valine

Leu: leucine

Ile: isoleucine

Ser: serine

Thr: threonine

Cys: cysteine

Met: methionine

Glu: glutamic acid

Asp: aspartic acid

Lys: lysine

Arg: arginine

His: histidine

Phe: phenylalanine

Tyr: tyrosine

Trp: tryptophan

Pro: proline

Asn: asparagine

Gln: glutamine

pGlu: pyroglutamic acid

Hse: homoserine

Also, substituents, protecting groups, and reagents frequently used in this specification are presented as the codes below.

Me: methyl group

Et: ethyl group

Bu: butyl group

Ph: phenyl group

TC: thiazolidine-4(R)-carboxamide group

Tos: p-toluenesulfonyl

CHO: formyl

Bzl: benzyl

Cl₂-Bzl: 2,6-dichlorobenzyl

Bom: benzyloxymethyl

Z: benzyloxycarbonyl

Cl-Z: 2-chlorobenzyl oxycarbonyl

Br-Z: 2-bromobenzyl oxycarbonyl

Boc: t-butoxycarbonyl

DNP: dinitrophenol

Trt: trityl

Bum: t-butoxymethyl

Fmoc: N-9-fluorenyl methoxycarbonyl

HOBt: 1-hydroxybenztriazole

HOOBt: 3,4-dihydro-3-hydroxy-4-oxo-1,2,3-benzotriazine

HONB: 1-hydroxy-5-norbornene-2,3-dicarboxyimide

DCC: N,N′-dichlorohexylcarbodiimide

The sequence identification numbers in the sequence listing of the specification indicates the following sequence, respectively.

[SEQ ID NO: 1]

This shows the amino acid sequence of Protein A of the present invention that controls the production of Aβ.

[SEQ ID NO: 2]

This shows the amino acid sequence of Protein B of the present invention that controls the production of Aβ.

[SEQ ID NO: 3]

This shows the amino acid sequence of Protein C of the present invention that controls the production of Aβ.

[SEQ ID NO: 4]

This shows the base sequence encoding Protein A of the present invention that controls the production of Aβ.

[SEQ ID NO: 5]

This shows the base sequence encoding Protein B of the present invention that controls the production of Aβ.

[SEQ ID NO: 6]

This shows the base sequence encoding Protein C of the present invention that controls the production of Aβ.

[SEQ ID NO: 7]

This shows the amino acid sequence of C53 used in EXAMPLE 1, wherein methionine is added at the N terminus of APP fragment from the N terminus of Aβ to the APP cell membrane region containing the γ-secretase cleavage site.

[SEQ ID NO: 8]

This shows the base sequence of the primer used in EXAMPLE 1.

[SEQ ID NO: 9]

This shows the base sequence of the primer used in EXAMPLE 1.

[SEQ ID NO: 10]

This shows the amino acid sequence of intracellular domain [NICD] of mouse Notch 1 used in EXAMPLE 1.

[SEQ ID NO: 11]

This shows the base sequence of the primer used in EXAMPLE 1.

[SEQ ID NO: 12]

This shows the base sequence of the primer used in EXAMPLE 1.

[SEQ ID NO: 13]

This shows the base sequence of the DNA oligomer used in EXAMPLE 1.

[SEQ ID NO: 14]

This shows the base sequence of the DNA oligomer used in EXAMPLE 1.

[SEQ ID NO: 15]

This shows the base sequence of HES-1 promoter described in EXAMPLE 2.

[SEQ ID NO: 16]

This shows the base sequence of the primer used in EXAMPLE 6.

[SEQ ID NO: 17]

This shows the base sequence of the primer used in EXAMPLE 6.

[SEQ ID NO: 18]

This shows the amino acid sequence of Aβ40.

[SEQ ID NO: 19]

This shows the amino acid sequence of Aβ42.

[SEQ ID NO: 20]

This shows the amino acid sequence of Aβ52.

[SEQ ID NO: 21]

This shows the base sequence of full-length 5-lipoxygenase cDNA.

[SEQ ID NO: 22]

This shows the amino acid sequence of full-length 5-lipoxygenase.

[SEQ ID NO: 23]

This shows the base sequence of the sense primer used in EXAMPLE 7 to amplify PCR product 1.

[SEQ ID NO: 24]

This shows the base sequence of the anti-sense primer used in EXAMPLE 7 to amplify PCR product 1.

[SEQ ID NO: 25]

This shows the base sequence of the sense primer used in EXAMPLE 7 to amplify PCR product 2.

[SEQ ID NO: 26]

This shows the base sequence of the anti-sense primer used in EXAMPLE 7 to amplify PCR product 1.

Transformant Escherichia coli DH5α/pCxNC53NICD bearing the plasmid pCxNC53NICD obtained in EXAMPLE 1 described below has been on deposit with International Patent Organisms Depository, National Institute of Advanced Industrial Science and Technology (formerly, National Institute of Bioscience and Human-Technology (NIBH), Ministry of International Trade and Industry), located at Central 6, 1-1-1 Higashi, Tsukuba, lbaraki, 305-8566, Japan, as the Accession Number FERM BP-7676 since Jul. 26, 2001 and with Institute for Fermentation (IFO), located at 2-17-85, Juso-honmachi, Yodogawa-ku, Osaka-shi, Osaka, 532-8686, Japan, as the Accession Number IFO 16651 on Jun. 19, 2001.

Transformant Escherichia coli DH5α/pHESpac bearing the plasmid pHESpac obtained in EXAMPLE 2 described below has been on deposit with International Patent Organisms Depository, National Institute of Advanced Industrial Science and Technology (formerly, National Institute of Bioscience and Human-Technology (NIBH), Ministry of International Trade and Industry), located at Central 6, 1-1-1 Higashi, Tsukuba, Ibaraki, 305-8566, Japan, as the Accession Number FERM BP-7677 since Jul. 26, 2001 and with Institute for Fermentation (IFO), located at 2-17-85, Juso-honmachi, Yodogawa-ku, Osaka-shi, Osaka, 532-8686, Japan, as the Accession Number IFO 16652 on Jun. 19, 2001.

### EXAMPLES

Hereinafter, the present invention will be described specifically with reference to EXAMPLES but is not deemed to be limited thereto. The gene manipulation procedures using Escherichia coli were performed according to the methods described in the Molecular Cloning.

### Example 1

### Preparation of DNA Encoding Chimeric Protein C53NICD

Using human APP cDNA (Kang J. et al., Nature, 32, 733-736, 1987) as a template, DNA encoding C53 (SEQ ID NO: 7), to which methionine was added at the N terminus of the APP fragment from the N terminus of APP to the APP cell membrane region containing the γ-secretase cleavage site, was prepared by PCR.

As a sense strand primer and an antisense strand primer, Oligo DNA (ATCTGGTACCCCACCATGGATGCAGAATTCCGACATGAC) represented by SEQ ID NO: 8 and oligo DNA (GCTTCTAGACAGCATCACCAAGGTGATGACGAT) represented by SEQ ID NO: 9 were used, respectively. In this preparation, the KpnI site and the XbaI site were introduced at the 5′ and 3′ ends, respectively, to prepare a chimeric DNA with the DNA fragment encoding intracellular domain (NICD) (SEQ ID NO: 10) of mouse Notch 1.

The DNA fragment encoding NICD was prepared by PCR in accordance with the following procedures, using as a template mouse Notch 1 cDNA (Amo FF et al., Genomics, 15, 259-264, 1993). First, a short DNA fragment (928 bp) encoding the partial sequence from the N terminus to the C terminus of NICD was prepared by PCR, using oligo DNA (CTGTCTAGAAAGCGCCGGCGCCAGCATGGCCAG) represented by SEQ ID NO: 11 as a sense strand primer and as an antisense strand primer oligo DNA (ATTGTTCACCGCGGCCGCCCAATG) represented by SEQ ID NO: 12. This fragment was artificially added with the XbaI site at the 5′ end, and the NotI site derived from mouse Notch 1 cDNA sequence was contained at the 3′ end.

Next, the DNA fragment encoding C53 was ligated thereto with XbaI. This chimeric DNA fragment was inserted at this site of pCxN (Niwa H et al., Gene 108:193, 1991), in which the KpnI and NotI sites had been previously introduced. Thus, plasmid pCxNC53NICDΔC containing the chimeric DNA encoding C53 and NICD to the partway at the C terminal side was prepared.

Next, in order to prepare the DNA fragment encoding the full-length NICD, the NotI-NotI DNA fragment was prepared from mouse Notch 1 cDNA Ssp I-Hind III fragment containing the NICD coding region, which had been previously cloned, and was inserted into the NotI site of pCxNC53NICDΔC. Thus, the chimeric DNA encoding C53NICD was prepared. The plasmid bearing this chimeric DNA was named pCxNC53NICD. Though pCxN (Niwa H. et al., Gene 108:193, 1991) used as a vector is a plasmid having a promoter of β-actin and a neomycin-resistant gene, KpnI and NotI sites were newly introduced into the plasmid by inserting the DNA oligomers represented by SEQ ID NO: 13 and SEQ ID NO: 14, respectively, at the EcoRI site of pCxN, and the resulting product was used to prepare pCxNC53NICD:

SEQ ID NO: 13
(5′-AATTCGGTACCCCCGGGGCGGCCGCCTCGAGGA-3′)
SEQ ID NO: 14
(3′-GCCATGGGGGCCCCGCCGGCGGAGCTCCTTTAA-5′)

### Example 2

### Preparation of Puromycin-resistant Gene (pac: puromycin-N-acetyl-transferase gene) having HES-1 as a Promoter

HES1 promoter (SEQ ID NO: 15) (Takebayashi K., et al., J. Biol. Chem. 269 (7):5150-6, 1994) was prepared by adding KpnI and HindIII sites, using mouse chromosome as a template, and amplifying by PCR. Next, the promoter was inserted into vector PGV-B (purchased from TOYO B-Net Co., Ltd.) at the KpnI and HindHIII sites. This plasmid was named pGV-B-HES-1. On the other hand, the puromycin-resistant gene was prepared from pPUR (Clontech, Inc.) by excising the DNA fragment coding for the puromycin-resistant gene with restriction enzymes HindIII and BamHI. The DNA fragment was inserted into pGV-B-HES-1 at the HindIII and BamHI sites, and the completed plasmid was named pHESpac.

### Example 3

### Establishment of Cell Line: A5-9 (and A5-9-PS1) which Permanently Expresses the Puromycin-resistant Gene and C53NICDcDNA (and human human presenilin 1 cDNA)

The constructed plasmid described above was transfected to mouse pro-B cell-derived cell line, BaF/3 cells (Palacios, R., et al., Cell, 41:727, 1985) by electroporation. Neomycin and puromycin resistance was used as an indicator to select cells. The transformant obtained was named A5-9 cell. Human presenilin 1 cDNA was further introduced into the A5-9 cell. Human presenilin 1 cDNA was prepared by PCR using cDNA prepared from human brain (Sudoh, S. et al., J. Neurochem. 71:1535, 1998) and inserted into a vector having SRα promoter (Takebe,Y., Mol. Cell. Biol. 8:466, 1988) and a hygromycin-resistant gene, which was then transfected to the A5-9 cell by electroporation. Hygromycin resistance was used as an indicator to select cells. The transformant obtained was named A5-9-PS1 cell.

### Example 4

### Preparation of Human cDNA Library and Transfection to A5-9 or A5-9-PS1 Cells

Human cDNA library was prepared from human hippocampus-derived mRNA (Clontech, Inc.) by synthesizing cDNA using a cDNA synthesis kit (SuperScript™ Choice system) commercially available from Gibco, Inc. The obtained cDNA, to which BstX I adapter (Invitrogen, Inc.) was added, was introduced into retrovirus vector pMX (Onishi, M. et al., Exp. Hematol. 24:324, 1996) at the BstXI site. This library was transfected to packaging cells, Phnenix-Eco cells to produce virus containing the human cDNA library (Xu X. et al., Nature Genetics. 27:23-29, 2001; Hitoshi Y. et al., Immunity. 8:461-471, 1998). The virus was infected to A5-9 cells or A5-9-PS1 cells (cell count of 4×10⁶, respectively) in accordance with the method reported before (Kitamura, K. et al., Proc. Natl. Acad. Sci., 92:9146, 1995). GFP-encoding cDNA encoding was co-infected and the cells wherein GFP was expressed were subjected to fluorometric analysis. The result indicates that the infection efficiency was about 25%.

### Example 5

### Selection of Cells to Increase the Production of Aβ

The A5-9 cells or A5-9-PS1 cells described in EXAMPLE 4, to which the human hippocampus cDNA library was transfected through the infection with retrovirus, were cultured in the presence of puromycin of a high concentration (A5-9 cells, 25 μg/ml; A5-9-PS1 cells, 9 μg/ml). Then, the cells which acquired puromycin resistance were selected. The concentrations of puromycin used were determined based on the respective minimum puromycin concentrations of 20 μg/ml and 5 μg/ml, at which the A5-9 cells and A5-9-PS1 cells as the parent strain were extinct. Next, Aβ produced by these cells was assayed by high sensitive western blotting (Ida N. et al., J. Biol. Chem. 271:22908, 1996). That is, Aβ was detected as follows. The Aβ secreted in a medium by incubation for 3 days (initial cell concentration of 2×10⁵ cells/ml, medium of 3 ml) was immunoprecipitated by anti-Aβ monoclonal antibody 6E10 and the deposits were detected by high sensitive western blotting using anti-Aβ antibody. Comparison for judgment in the amount of Aβ produced was performed by comparing the intensity of the band of Aβ detected by western blotting to the amount of Aβ produced by the parent strain. The results are shown in TABLE 1. In the A5-9 cells where PS1 was not overexpressed, 16 clones of the puromycin-resistant cells were acquired and in the A5-9-PS1 cells where PSI was overexpressed, about 60 clones in total of the puromycin-resistant cells were acquired after transfection twice. Among them, it was revealed that 5 clones in the A5-9 cells and 25 clones in A5-9-PS1 increased the production of Aβ40 (TABLE 1).

### Example 6

### Identification of cDNA that Increases the Production of Aβ

From the clones selected in EXAMPLE 5 above to increase the production of Aβ, human hippocampus-derived cDNA was amplified by PCR using virus vector pMX sites as primers (which were identified to be sense strand primer, SEQ ID NO: 16; GGTGGACCATCCTCTAGACTG and antisense strand primer, SEQ ID NO: 17; GTTACTTAAGCTAGCTTGCC). The cDNA obtained was ligated with vector pcDNA3 (Invitrogen, Inc.), which was transfected to 4×10⁵ cells of HEK293 cells (Tomita S., et al., J. Biol. Chem. 273: 19304-19310, 1998; Tomita S., et al., J. Biol. Chem. 275: 13056-13060, 2000) where APP and PS1 were overexpressed. One day after, the medium was changed to a new medium followed by incubation for 24 hours. The amount of Aβ selected in the medium was assayed by ELISA (Asami-Odaka, A. et al., 34:10272, 1995). It was found so far that 3 kinds of human hippocampus-derived cDNAs increased the amount of Aβ produced (TABLE 2). One of them was identified to be identical with human cDNA (accession No. AAH06223) registered in Genbank. In the following procedures of the present invention, this cDNA was named gene A (SEQ ID NO: 4) and the protein encoding the cDNA was named Protein A (SEQ ID NO: 1). Gene A showed extremely high homology to cDNA (accession No. AK003241), which was registered as mouse cDNA encoding a protein with unknown function. Moreover, Gene A showed high homology also to Syndesmos (Baciu P. C., et al., J. Cell Science, 113, 315, 2000; accession no. AF095446), which was identified from chicken to be a protein capable of binding to syndecan-4 by the yeast two-hybrid technique. The one found to be a gene to control the production of Aβ was a protein, Herp (Kokame K. et al., J. Biol. Chem. 275: 3286, 2000; accession no. AB034989) (cDNA: SEQ ID NO: 5, protein: SEQ ID NO: 2), which was localized in the endoplasmic reticulum and its expression was induced by endoplasmic reticulum stress but its function was unknown. Further the other one found to be a gene to control the production of Aβ was cDNA (cDNA: SEQ ID NO: 6, protein: SEQ ID NO: 3) containing the 1-389 internal sequence from the N terminus of 5-lipoxygenase (accession no. XM 005818). Based on these results, it is likely that the three genes described above and their products would take part in the development and progression of Alzheimer's disease.

TABLE 1
Summary of the number of puromycin-resistant clones
acquired by infection with retrovirus of human cDNA
library and the number of clones to increase the
production of Aβ40

| | | | Number of clones |
|---|---|---|---|
| Parent | Concentration of | Number of | which increased |
| Strain | puromycin (mg/ml) | clones survived | Aβ production |
| A5-9-PS1² | 9 | 32(5)¹ | 12 |
| A5-9-PS1³ | 9 | 45(14) | 13 |
| A5-9 | 25 | 18(2) | 5 |

Human hippocampus-derived cDNA library was transfected to 4×10⁶ cells by the transfection technique using a retrovirus.

1 The numerical figure within parenthesis shows the number of clones for control, which were survived when the cDNA library was not infected.

2, 3 The results of selection in 2 runs are shown.

TABLE 2
cDNA found to increase the amount of Aβ produced from the full-length APP

| | Aβ40 | | Aβ42 cDNA | |
|---|---|---|---|---|
| | | Increasing | | Increasing |
| | Concentration | rate of Aβ42 | Concentration | rate of Aβ40 |
| | [pM] | produced | [pM] | produced |
| Protein A | 378 ± 10 (232 ± 34) | 1.6 | 88 ± 22 (45 ± 2) | 2.0 |
| Herp | 631 ± 20 (455 ± 26) | 1.4 | 222 ± 16 (146 ± 11) | 1.5 |
| 5-Lipoxygenase | 652 ± 27 (455 ± 26) | 1.5 | 272 ± 13 (146 ± 11) | 1.9 |

The numerical figure within parenthesis shows the amount of Aβ produced when the vector alone was transfected for control, wherein the data shows a mean value of 2 samples and the increasing rate of Aβ produced shows a rate of increase when compared to control.

### Example 7

Also with regard to the partial cDNA sequence (SEQ ID NO: 6) of 5-lipoxygenase obtained by screening in EXAMPLE 6, the full-length 5-lipoxygenase (cDNA is represented by SEQ ID NO: 21 and the amino acid sequence is represented by SEQ ID NO: 22) was prepared from the human hippocampus-derived cDNA library described in EXAMPLE 4 by PCR, as described below. That is, PCR Product 1 (EcoRI site-added sense primer CGGAATTCCGCGCCATGCCCTCCTACACG (SEQ ID NO: 23); antisense primer CCCCGCATGCCGTACACGTAGACA (SEQ ID NO: 24)) and PCR Product 2 (SalI site-added sense primer TGTCTACGTGTACGGCATGCGGGG (SEQ ID NO: 25); antisense primer GCGTCGACCTGGCTGGGGCAGCTGGCCTTCCC (SEQ ID NO: 26)) were amplified by PCR, respectively, using as a template the human hippocampus-derived cDNA library. The two PCR products obtained by PCR were subjected to ligation at the SphI site to prepare the full-length 5-lipoxygenase cDNA. Using this full-length 5-lipoxygenase cDNA, the effect on Aβ production was examined. The results are shown in TABLE 3.

In TABLE 3, control shows the cells transfected with the virus vector alone (pMX: described in EXAMPLE 4). Immortalized mouse fibroblasts of 2×10⁵ together with human APP cDNA were intracellularly transfected by infection of the cDNA with retrovirus (described in EXAMPLE 4). One day after, a new medium was replaced and the amount of Aβ secreted in the medium for 4 days was assayed by ELISA. The increasing rate of Aβ produced is shown by a rate of increase when compared to control, wherein the data are shown in a mean value of 2 samples.

TABLE 3
Effect of expression of the full-length 5-lipoxygenase
cDNA on Aβ production

| | Increasing rate of | Increasing rate of |
|---|---|---|
| cDNA | Aβ40 produced | Aβ42 produced |
| Control | 1.0 | 1.0 |
| 5-Lipoxygenase | 2.6 ± 0.2 | N.D. |

N.D.: not determined (not assayed)

### Industrial Applicability

According to the present invention, the method of screening genes that control the production of Aβ is provided and Alzheimer's disease-associated genes or Alzheimer's disease-associated candidate genes are provided. The present invention provides these genes, nucleotides that specifically hybridize to these genes, transformants by recombinant vectors bearing these genes, proteins encoded by these cDNAs, or the methods of diagnosis, treatment and prevention of Alzheimer's disease using antibodies to the proteins. The present invention further provides these genes, nucleotides that specifically hybridize to these genes, transformants by recombinant vectors bearing these genes, proteins encoded by these cDNAs, or the method of screening Aβ production inhibitors using antibodies to the proteins, the Aβ production inhibitors obtained by the screening method, and the methods of diagnosis, treatment and prevention of Alzheimer's disease using the Aβ production inhibitors.

1. A method of screening a DNA that controls the production of amyloid β protein (Aβ), which comprises transfecting a DNA library to the cell line designed to activate the expression of a selection marker gene by the increased production of Aβ from the Aβ precursor protein (βAPP) fragment, and identifying a DNA clone transfected with the cell in which the production of Aβ is increased.

2. The screening method according to claim 1, wherein the DNA that controls the production of Aβ is human cDNA.

3. The screening method according to claim 1, wherein the DNA that controls the production of Aβ is human chromosomal DNA.

4. The screening method according to claim 1, wherein the cell line is a transformant transformed by (1) a vector bearing a DNA encoding a fusion protein of the βAPP fragment containing the γ-secretase cleavage site of βAPP and a transcription-promoting factor, and (2) a vector bearing a DNA ligated to the selection marker gene downstream of the promoter to induce transcription of the selection marker gene by the transcription activity of transcription-promoting factor.

5. The screening method according to claim 1, wherein the cell line is a transformant transformed by (1) a vector bearing a DNA encoding a fusion protein (CAPP-NICD) of the βAPP fragment containing the γ-secretase cleavage site of βAPP and the C-terminal transcription factor domain of Notch, and (2) a vector bearing a DNA ligated to a drug-resistant gene downstream of HES-1 promoter to induce transcription of drug-resistant gene by the transcription activity of Notch.

6. The screening method according to claim 4 or 5, wherein the cell line co-expresses the fusion protein and the selection marker.

7. A DNA containing a DNA hybridizable to the DNA that controls the production of amyloid β protein (Aβ) under high stringent conditions which is obtainable by the screening method according to claim 1.

8. The DNA according to claim 7, wherein the DNA that controls the production of Aβ is human cDNA.

9. The DNA according to claim 7, wherein the DNA that controls the production of Aβ is human chromosomal DNA.

10. The DNA according to claim 7, which is associated with Alzheimer's disease.

11. The DNA according to claim 7, which is a DNA that increases the production of Aβ.

12. The DNA according to claim 11, wherein the DNA that increases the production of Aβ is a DNA encoding human cDNA (Genbank accession No. AAH06223) containing the base sequence represented by SEQ ID NO: 4.

13. The DNA according to claim 11, wherein the DNA that increases the production of Aβ is a DNA encoding human Herp (Genbank accession No. AB034989) containing the base sequence represented by SEQ ID NO: 5.

14. The DNA according to claim 11, wherein the DNA that increases the production of Aβ is a DNA encoding human 5-lipoxygenase (Genbank accession no. XM 005818) containing the base sequence represented by SEQ ID NO: 6.

15. The DNA according to claim 11, wherein the DNA that increases the production of Aβ is a DNA encoding the full-length sequence of human 5-lipoxygenase containing the base sequence represented by SEQ ID NO: 21.

16. A recombinant vector comprising the DNA according to claim 7.

17. A transformant transformed by the recombinant vector according to claim 16.

18. A process of manufacturing a peptide or protein encoded by the DNA according to claim 7, or a salt thereof, which comprises culturing the transformant according to claim 17 and producing the peptide or protein encoded by the DNA according to claim 7.

19. A diagnostic product comprising the DNA according to claim 7 or a part thereof.

20. The diagnostic product according to claim 19, which is a diagnostic product for Alzheimer's disease.

21. A method for diagnosis of Alzheimer's disease, which comprises using the DNA according to claim 7.

22. A method for detection of single nucleotide polymorphisms (SNPs), which comprises using the DNA according to claim 7.

23. The method for detection according to claim 22, which comprises decoding the base sequence of a DNA corresponding to the DNA according to claim 7 of a patient with Alzheimer's disease and comparing the decoded base sequence with that of the DNA according to claim 7.

24. Single nucleotide polymorphisms (SNPs) of the DNA according to claim 7.

25. A diagnostic product comprising the single nucleotide polymorphisms (SNPs) according to claim 24.

26. The diagnostic product according to claim 25, which further contains the DNA according to claim 7 or a part thereof.

27. The diagnostic product according to claim 25 or 26, which is a diagnostic product for Alzheimer's disease.

28. A method for diagnosis of Alzheimer's disease, which comprises using single nucleotide polymorphisms (SNPs) according to claim 24.

29. The method for diagnosis according to claim 28, wherein the DNA according to claim 7 or a part thereof is used.

30. A peptide or protein encoded by the DNA according to claim 7, or a salt thereof.

31. The peptide, protein or its salt according to claim 30, which is associated with Alzheimer's disease.

32. The peptide, protein or its salt according to claim 30, which increases the production of Aβ.

33. A protein that increases the production of Aβ, or a salt thereof, containing the same or substantially the same amino acid sequence as the amino acid sequence represented by SEQ ID NO: 1.

34. A protein that increases the production of Aβ, or a salt thereof, containing the same or substantially the same amino acid sequence as the amino acid sequence represented by SEQ ID NO: 2.

35. A protein that increases the production of Aβ, or a salt thereof, containing the same or substantially the same amino acid sequence as the amino acid sequence represented by SEQ ID NO: 3.

36. A protein that increases the production of Aβ, or a salt thereof, containing the same or substantially the same amino acid sequence as the amino acid sequence represented by SEQ ID NO: 22.

37. An antibody to the peptide, protein or its salt according to any one of claims 29 through 36.

38. The antibody according to claim 37, which is a neutralizing antibody for inactivating the activity of the peptide, protein or its salt according to any one of claims 29 through 36.

39. A diagnostic product comprising the antibody according to claim 37.

40. The diagnostic product according to claim 39, which is a diagnostic product for Alzheimer's disease.

41. A method for quantification of the peptide, protein or its salt according to any one of claims 29 through 36, which comprises using the antibody according to claim 37.

42. A method for diagnosis of Alzheimer's disease, using the method for quantification according to claim 41.

43. A pharmaceutical comprising an antibody to the peptide, protein or its salt according to claim 32, which increases the production of Aβ.

44. The pharmaceutical according to claim 43, which is a preventive/therapeutic agent for Alzheimer's disease.

45. A method of preventing/treating Alzheimer's disease, which comprises administering to a mammal an effective dose of the antibody to the peptide, protein or its salt according to claim 32 that increases the production of Aβ.

46. An antisense DNA comprising a complementary base sequence to the DNA according to claim 7, or a part thereof.

47. A diagnostic product comprising the antisense DNA according to claim 46.

48. The diagnostic product according to claim 47, which is a diagnostic product for Alzheimer's disease.

49. A pharmaceutical comprising an antisense DNA comprising a complementary base sequence to the DNA according to claim 11 that increases the production of Aβ, or a part thereof.

50. The pharmaceutical according to claim 49, which is a preventive/therapeutic agent for Alzheimer's disease.

51. A method of preventing/treating Alzheimer's disease, which comprises administering to a mammal an effective dose of the antisense DNA according to claim 49.

52. A method of screening an amyloid β protein (Aβ) production inhibitor, which comprises using the DNA according to claim 7.

53. A method of screening an amyloid β protein (Aβ) production inhibitor, which comprises using the peptide, protein or its salt according to claim 30.

54. A method of screening an amyloid β protein (Aβ) production inhibitor, which comprises using the antibody according to claim 37.

55. A method of screening an amyloid β protein (Aβ) production inhibitor, which comprises using the antisense DNA according to claim 46.

56. A method of screening an amyloid β protein (Aβ) production inhibitor, which comprises using the transformant according to claim 17.

57. An amyloid β protein (Aβ) production inhibitor, which is obtainable by the screening method according to any one of claims 52 through 56.

58. A pharmaceutical comprising the Aβ production inhibitor according to claim 57.

59. The pharmaceutical according to claim 58, which is a preventive/therapeutic agent for Alzheimer's disease.

60. A method of preventing/treating Alzheimer's disease, which comprises administering to a mammal an effective dose of the Aβ production inhibitor according to claim 57.

61. A method of screening a substance that controls the production of an amyloid β protein (Aβ), which comprises measuring a difference between respective levels of Aβ produced, when a test compound is added to (i) the cell line designed to activate the expression of a selection marker gene by the increased production of Aβ from the Aβ precursor protein (βAPP) fragment, and to (ii) the cell line transfected with the DNA that increases the production of Aβ according to claim 11.

62. A method of screening a substance that controls the production of amyloid β protein (Aβ), which comprises measuring a difference between respective biological activities of the selection marker, when a test compound is added to (i) the cell line designed to activate the expression of a selection marker gene by the increased Aβ production from the Aβ precursor protein (βAPP) fragment, and to (ii) the cell line transfected with the DNA that increases the production of Aβ according to claim 12.

63. The screening method according to claim 62, wherein the selection marker gene is a drug-resistant gene and the biological activity of the selection marker is drug resistance.

64. A method of screening an expression inhibitor of the DNA, which comprises using the DNA according to claim 7.

65. A method of screening a compound that suppresses or promotes the promoter activity of a gene encoding the peptide or protein, which comprises assaying the respective reporter activities in combination of the promoter region in the human chromosomal DNA according to claim 9 with a reporter gene, where a test compound is added and where no test compound is added.

66. A method of screening a compound that suppresses the respective expression levels of said peptide, protein or its salt according to claim 30, or DNAs thereof, when a test compound is added to a cell capable of expressing the said peptide, protein or its salt, and when no test compound is added thereto.
